(19) United States
(12) Patent Application Publication
(10) Pub. No.: US 2013/0296345 A1
(43) Pub. Date: Nov. 7, 2013

(54) METHODS FOR TREATING HYPERURICEMIA AND RELATED DISEASES

(71) Applicant: Barry D. Quart, Encinitas, CA (US)

(72) Inventor: Barry D. Quart, Encinitas, CA (US)

(73) Assignee: Ardea Biosciences, Inc., San Diego, CA (US)

(21) Appl. No.: 13/879,373

(22) PCT Filed: Oct. 15, 2010
(86) PCT No.: PCT/US10/52958

§ 371 Date: May 1, 2013

(51) Int. Cl. A61K 31/4196; A61K 31/426; A61K 31/519

(52) U.S. Cl. CPC A61K 31/4196; A61K 31/519; A61K 31/426
USPC 514/262.1; 514/365; 514/384

(57) ABSTRACT

Provided herein are methods of treating gout, treating hyperuricemia, lowering serum uric acid, or the like with compounds of formula (I) have the following structure Further, provided herein are compositions comprising a compound of formula (I).

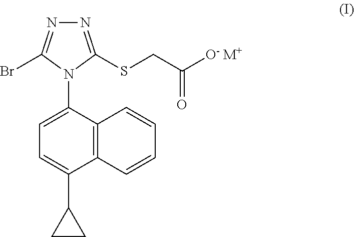

### BACKGROUND OF THE INVENTION

Gout is a condition that results from uric acid crystals depositing in tissues of the body. It is often related to an inherited abnormality in the body's ability to process uric acid, but may also be exacerbated by a diet high in purines. Defective uric acid processing may lead to elevated levels of uric acid in the blood causing recurring attacks of joint inflammation (arthritis), uric acid deposits in and around the joints, decreased kidney function, and kidney stones. Approximately 3-5 million people in the United States suffer from attacks of gout with attacks 6 to 9 times more common in men than in women (see Sanders and Wortmann, “Harrison's Principles of Internal Medicine”, 16th Edition; 2005; Food and Drug Administration (FDA) Advisory Committee Meeting, Terkeltaub presentation, June 2004; Terkeltaub, “Gout”, N Engl J Med., 349, 1647-55, 2003).

### SUMMARY OF THE INVENTION

Provided in certain embodiments herein is a method of treating gout or hyperuricemia in a subject, wherein the gout is refractory, non-responsive, and/or resistant to a monotherapy with an agent other than a compound of formula (I), the method comprising administering to the subject a therapeutically effective amount of a compound of formula (I).

In some embodiments, the gout or hyperuricemia is refractory, non-responsive, and/or resistant to allopurinol monotherapy. In specific embodiments, described herein are methods of treating gout in a subject wherein the subject has received treatment with allopurinol and wherein the allopurinol treatment does not decrease serum uric acids levels below about 6 mg/dL. In some embodiments, such methods comprise administering a therapeutically effective amount of a compound of formula (I). In specific embodiments, the methods comprise administering to the subject allopurinol and a compound of formula (I).

In some embodiments, the gout or hyperuricemia is refractory, non-responsive, and/or resistant to febuxostat monotherapy. In specific embodiments, described herein are methods of treating gout in a subject wherein the subject has received treatment with febuxostat and wherein the febuxostat treatment does not decrease serum uric acids levels below about 6 mg/dL. In some embodiments, such methods comprise administering a therapeutically effective amount of a compound of formula (I). In specific embodiments, the methods comprise administering to the subject febuxostat and a compound of formula (I).

Compounds of formula (I) have the following structure

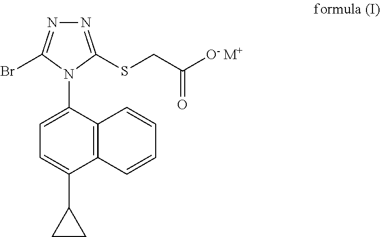

wherein M is H, Na, Ca, Mg, Zn, K, Al, piperazine or meglumine

In some embodiments, after administration of the compound of formula (I) and, optionally, allopurinol, the serum uric acids levels of the subject decrease below about 6 mg/dL. In specific embodiments, after administration of allopurinol and the compound of formula (I) the serum uric acids levels of the subject decrease below about 6 mg/dL.

In other embodiments, after administration of the compound of formula (I) and, optionally, febuxostat, the serum uric acids levels of the subject decrease below about 6 mg/dL. In specific embodiments, after administration of febuxostat and the compound of formula (I) the serum uric acids levels of the subject decrease below about 6 mg/dL.

In certain embodiments, a method described herein comprises administering any suitable amount (e.g., an effective amount) of a compound of formula (I), e.g., to an individual in need thereof. In some embodiments, from about 50 mg to about 1000 mg of the compound of formula (I) is administered. In certain embodiments, from about 100 mg to about 1000 mg of the compound of formula (I) is administered. In other embodiments, from about 100 mg to about 800 mg of the compound of formula (I) is administered. In some embodiments, from about 100 mg to about 600 mg of the compound of formula (I) is administered. In further or additional embodiments, from about 100 mg to about 400 mg of the compound of formula (I) is administered. In some embodiments, about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg of a compound of formula (I) is administered.

In further or additional embodiments, a method described herein comprises administering any suitable amount (e.g., an effective amount, such as alone or in combination with a compound of formula (I)) of allopurinol, e.g., to an individual in need thereof. In some embodiments, from about 100 mg to about 1000 mg of allopurinol is administered. In other embodiments, from about 100 mg to about 800 mg of allopurinol is administered. In some embodiments, from about 100 to about 600 mg of allopurinol is administered. In certain embodiments, from about 200 mg to about 500 mg of allopurinol is administered. In some embodiments, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg of allopurinol is administered. In further or additional embodiments, from about 100 mg to about 600 mg of the compound of formula (I) and from about 200 mg to about 500 mg of allopurinol is administered.

In further or additional embodiments, a method described herein comprises administering any suitable amount (e.g., an effective amount, such as alone or in combination with a compound of formula (I)) of febuxostat, e.g., to an individual in need thereof. In some embodiments, from about 20 mg to about 200 mg of febuxostat is administered. In further or additional embodiments, from about 30 mg to about 150 mg of febuxostat is administered. In certain embodiments, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, or about 200 mg of febuxostat is administered. In further or additional embodiments, from about 100 mg to about 600 mg of the compound of formula (I) and about 40 mg of febuxostat is administered. In further or additional embodiments, from about 100 mg to about 600 mg of the compound of formula (I) and about 80 mg of febuxostat is administered. In further or additional embodiments, from about 100 mg to about 600 mg of the compound of formula (I) and about 120 mg of febuxostat is administered.

In some embodiments M is H or Na. In further or additional embodiments, M is Na. In further or additional embodiments, M is H. In further or additional embodiments, M is not Na. In further or additional embodiments, M is not H. In some embodiments, M is Ca (e.g., wherein Ca has a charge of ++). It is to be understood that, in certain instances, if M is Ca having a 2+ charge, the compound of formula (I) has the structure:

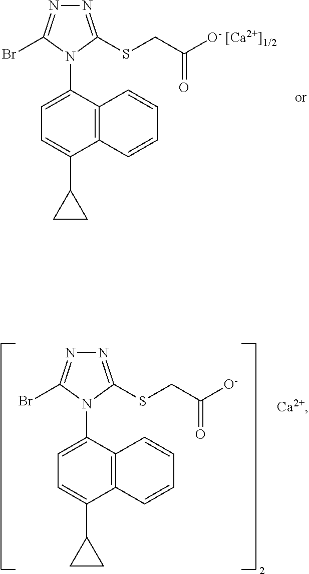

each of which are considered to be equivalent for the purposes of this disclosure. Other multiple charged cations (M) may also be used, e.g., Mg, Al, Zn, or the like (in each of these cases, the stoichiometric ratio of acid to M is such that the ionic charges are balanced (or another anion may be present to balance the excess cationic charge)). Therefore, in some embodiments, M is Ca and M⁺ is [Ca²⁺]_{1/2}, or M is Mg and M⁺ is [Mg²⁺]_{1/2}, or M is Al and M⁺ is [Al³⁺]_{1/3} or [Al²⁺]_{1/2} or Al+, or M is Zn and M⁺ is [Zn²⁺]_{1/2}.

In specific embodiments, M is H. In such embodiments, the interaction between the O— group and the H+ group may be an ionic interaction or a covalent bond. In certain embodiments, a compound of formula (I) has the structure:

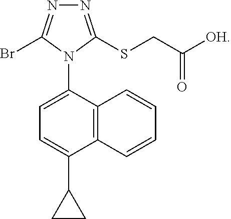 [SMILES: O=C(O)CSc1nnc(Br)n1-c1ccc(C2CC2)c2ccccc12]

In some embodiments, provided herein are methods for decreasing uric acid levels in one or more tissues, joints, organs or blood of a subject with elevated uric acid levels (e.g., a subject diagnosed with or suspected of having gout), the elevated uric acid levels being refractory, non-responsive, or resistant to allopurinol monotherapy, febuxostat monotherapy, PNP-inhibitor monotherapy, probenecid monotherapy, tranilast monotherapy, sulfinpyrazone monotherapy, losartan monotherapy, fenofibrate monotherapy, and/or benzbromarone monotherapy, by administering a therapeutically effective amount of a compound of formula (I). Provided in certain embodiments herein is a method of treating gout or hyperuricemia in a subject, wherein the gout is refractory, non-responsive, or resistant to allopurinol monotherapy, febuxostat monotherapy, PNP-inhibitor monotherapy, probenecid monotherapy, tranilast monotherapy, sulfinpyrazone monotherapy, losartan monotherapy, fenofibrate monotherapy, and/or benzbromarone monotherapy, the method comprising administering to the subject a therapeutically effective amount of a compound of formula (I). In certain embodiments, the therapy further comprises administering the agent to which the gout is resistant, the agent being administering in an amount that is therapeutically effective in combination with the compound of formula (I). In various embodiments, the compound of formula (I) is administered in any therapeutic amount described herein.

Provided in specific embodiments herein is a method of treating gout or hyperuricemia in a subject, wherein monotherapy of the gout or hyperuricemia with an agent other than a compound of formula (I) initially (e.g., after one week) reduces serum uric acid levels to below 6 mg/dL, but serum uric acid levels subsequently rise above 6 mg/dL, and wherein the method comprises administering to the subject a therapeutically effective amount of a compound of formula (I). In further embodiments, the method further comprises administering a second agent (e.g., any second agent described herein, such as allopurinol or febuxostat).

Also provided herein are methods for decreasing uric acid levels in one or more tissues, joints, organs or blood of a subject in need of decreased uric acid levels, comprising administering to the subject allopurinol and a compound of formula (I). In some embodiments, allopurinol and the compound of formula (I) are administered at the same time. In further or additional embodiments, allopurinol and the compound of formula (I) are administered at different times. In various embodiments, the compound of formula (I) and allopurinol are administered in any amount described herein. In further or additional embodiments, from about 100 mg to about 1000 mg of allopurinol is administered. In further or additional embodiments, from about 200 mg to about 500 mg of allopurinol is administered.

Also provided herein are methods for decreasing uric acid levels in one or more tissues, joints, organs or blood of a subject in need of decreased uric acid levels, comprising administering to the subject febuxostat and a compound of formula (I). In some embodiments, febuxostat and the compound of formula (I) are administered at the same time. In further or additional embodiments, febuxostat and the compound of formula (I) are administered at different times. In various embodiments, the compound of formula (I) and febuxostat are administered in any amount described herein. In further or additional embodiments, from about 20 mg to about 150 mg of febuxostat is administered. In further or additional embodiments, about 40 mg of febuxostat is administered. In further or additional embodiments, about 80 mg of febuxostat is administered. In further or additional embodiments, about 120 mg of febuxostat is administered.

Also provided herein are methods for decreasing uric acid levels in one or more tissues, joints, organs or blood of a subject in need of decreased uric acid levels, comprising administering to the subject febuxostat and a compound of formula (I). In some embodiments, febuxostat and the compound of formula (I) are administered at the same time. In further or additional embodiments, febuxostat and the compound of formula (I) are administered at different times. In various embodiments, the compound of formula (I) and febuxostat are administered in any amount described herein.

Also provided herein are methods for decreasing uric acid levels in one or more tissues, joints, organs or blood of a subject in need of decreased uric acid levels, comprising administering to the subject a PNP-inhibitor and a compound of formula (I). In various embodiments, the PNP-inhibitor and compound of formula (I) are administered in any suitable amount, such as an effective amount, or any amount described herein. In some embodiments, a PNP-inhibitor and the compound of formula (I) are administered at the same time. In further or additional embodiments, a PNP-inhibitor and the compound of formula (I) are administered at different times. In some embodiments, the PNP-inhibitor is 7-(((3R,4R)-3-hydroxy-4-(hydroxymethyl)pyrrolidin-1-yl)methyl)-3H-pyrrolo[3,2-d]pyrimidin-4(5H)-one (BCX4208):

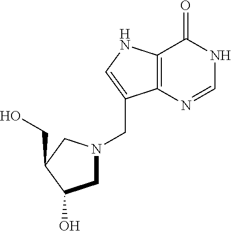

In further or additional embodiments, a method described herein comprises administering any suitable amount (e.g., an effective amount, such as alone or in combination with a compound of formula (I)) of BCX4208, e.g., to an individual in need thereof. In some embodiments, from about 10 mg to about 200 mg of BCX4208 is administered. In other embodiments, from about 20 mg to about 80 mg of BCX4208 is administered. In some embodiments, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, or about 200 mg of BCX4208 is administered. In further or additional embodiments, from about 100 mg to about 600 mg of the compound of formula (I) and from about 20 mg to about 80 mg of BCX4208 is administered.

Also provided herein are methods for decreasing uric acid levels in one or more tissues, joints, organs or blood of a subject in need of decreased uric acid levels, comprising administering to the subject probenecid and a compound of formula (I). In some embodiments, probenecid and the compound of formula (I) are administered at the same time. In further or additional embodiments, probenecid and the compound of formula (I) are administered at different times. In various embodiments, probenecid and compound of formula (I) are administered in any suitable amount, such as an effective amount, or any amount described herein. In further or additional embodiments, a method described herein comprises administering any suitable amount (e.g., an effective amount, such as alone or in combination with a compound of formula (I)) of probenecid, e.g., to an individual in need thereof. In some embodiments, from about 200 mg to about 3000 mg of probenecid is administered. In other embodiments, from about 250 mg to about 2000 mg of probenecid is administered. In some embodiments, from about 500 to about 2000 mg of probenecid is administered. In certain embodiments, about 200 mg, about 250 mg, about 300 mg, about 400 mg, about 500 mg, about 750 mg, about 1000 mg, about 1250 mg, about 1500 mg, about 1750 mg, about 2000 mg, about 2250 mg, about 2500 mg, about 2750 mg, or about 3000 mg of probenecid is administered. In further or additional embodiments, from about 100 mg to about 600 mg of the compound of formula (I) and from about 500 mg to about 2000 mg of probenecid is administered.

Also provided herein are methods for decreasing uric acid levels in one or more tissues, joints, organs or blood of a subject in need of decreased uric acid levels, comprising administering to the subject tranilast and a compound of formula (I). In some embodiments, tranilast and the compound of formula (I) are administered at the same time. In further or additional embodiments, tranilast and the compound of formula (I) are administered at different times. In various embodiments, tranilast and compound of formula (I) are administered in any suitable amount, such as an effective amount, or any amount described herein. In further or additional embodiments, a method described herein comprises administering any suitable amount (e.g., an effective amount, such as alone or in combination with a compound of formula (I)) of tranilast, e.g., to an individual in need thereof. In some embodiments, from about 50 mg to about 1500 mg of tranilast is administered. In other embodiments, from about 100 mg to about 1000 mg of tranilast is administered. In certain embodiments, from about 300 mg to about 900 mg of tranilast is administered. In some embodiments, about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 1000 mg, about 1100 mg, about 1200 mg, about 1300 mg, about 1400 mg, or about 1500 mg of tranilast is administered. In further or additional embodiments, from about 100 mg to about 600 mg of the compound of formula (I) and from about 300 mg to about 900 mg of tranilast is administered.

Also provided herein are methods for decreasing uric acid levels in one or more tissues, joints, organs or blood of a subject in need of decreased uric acid levels, comprising administering to the subject sulfinpyrazone and a compound of formula (I). In some embodiments, sulfinpyrazone and the compound of formula (I) are administered at the same time. In further or additional embodiments, sulfinpyrazone and the compound of formula (I) are administered at different times. In various embodiments, sulfinpyrazone and compound of formula (I) are administered in any suitable amount, such as an effective amount, or any amount described herein. In further or additional embodiments, a method described herein comprises administering any suitable amount (e.g., an effective amount, such as alone or in combination with a compound of formula (I)) of sulfinpyrazone, e.g., to an individual in need thereof. In some embodiments, from about 50 mg to about 1000 mg of sulfinpyrazone is administered. In other embodiments, from about 100 mg to about 800 mg of sulfinpyrazone is administered. In some embodiments, about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg of sulfinpyrazone is administered. In further or additional embodiments, from about 100 mg to about 600 mg of the compound of formula (I) and from about 100 mg to about 800 mg of sulfinpyrazone is administered.

Also provided herein are methods for decreasing uric acid levels in one or more tissues, joints, organs or blood of a subject in need of decreased uric acid levels, comprising administering to the subject losartan and a compound of formula (I). In some embodiments, losartan and the compound of formula (I) are administered at the same time. In further or additional embodiments, losartan and the compound of formula (I) are administered at different times. In various embodiments, losartan and compound of formula (I) are administered in any suitable amount, such as an effective amount, or any amount described herein. In further or additional embodiments, a method described herein comprises administering any suitable amount (e.g., an effective amount, such as alone or in combination with a compound of formula (I)) of losartan, e.g., to an individual in need thereof. In some embodiments, from about 10 mg to about 200 mg of losartan is administered. In other embodiments, from about 25 mg to about 100 mg of losartan is administered. In some embodiments, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, or about 200 mg of losartan is administered. In further or additional embodiments, from about 100 mg to about 600 mg of the compound of formula (I) and from about 25 mg to about 100 mg of losartan is administered.

Also provided herein are methods for decreasing uric acid levels in one or more tissues, joints, organs or blood of a subject in need of decreased uric acid levels, comprising administering to the subject fenofibrate and a compound of formula (I). In some embodiments, fenofibrate and the compound of formula (I) are administered at the same time. In further or additional embodiments, fenofibrate and the compound of formula (I) are administered at different times. In various embodiments, fenofibrate and compound of formula (I) are administered in any suitable amount, such as an effective amount, or any amount described herein. In further or additional embodiments, a method described herein comprises administering any suitable amount (e.g., an effective amount, such as alone or in combination with a compound of formula (I)) of fenofibrate, e.g., to an individual in need thereof. In some embodiments, from about 25 mg to about 250 mg of fenofibrate is administered. In other embodiments, from about 48 mg to about 145 mg of fenofibrate is administered. In some embodiments, about 25 mg, about 48 mg, about 50 mg, about 60 mg, about 70 mg, about 80 mg, about 90 mg, about 100 mg, about 110 mg, about 120 mg, about 130 mg, about 140 mg, about 145 mg, about 150 mg, about 160 mg, about 170 mg, about 180 mg, about 190 mg, about 200 mg, about 210 mg, about 220 mg, about 230 mg, about 240 mg, or about 250 mg of fenofibrate is administered. In further or additional embodiments, from about 100 mg to about 600 mg of the compound of formula (I) and from about 48 mg to about 145 mg of fenofibrate is administered.

Also provided herein are methods for decreasing uric acid levels in one or more tissues, joints, organs or blood of a subject in need of decreased uric acid levels, comprising administering to the subject benzbromarone and a compound of formula (I). In some embodiments, benzbromarone and the compound of formula (I) are administered at the same time. In further or additional embodiments, benzbromarone and the compound of formula (I) are administered at different times. In various embodiments, benzbromarone and compound of formula (I) are administered in any suitable amount, such as an effective amount, or any amount described herein. In further or additional embodiments, a method described herein comprises administering any suitable amount (e.g., an effective amount, such as alone or in combination with a compound of formula (I)) of benzbromarone, e.g., to an individual in need thereof. In some embodiments, from about 10 mg to about 500 mg of benzbromarone is administered. In other embodiments, from about 50 mg to about 200 mg of benzbromarone is administered. In some embodiments, about 10 mg, about 25 mg, about 50 mg, about 75 mg, about 100 mg, about 125 mg, about 150 mg, about 175 mg, about 200 mg, about 250 mg, about 300 mg, about 350 mg, about 400 mg, about 450 mg, or about 500 mg of benzbromarone is administered. In further or additional embodiments, from about 100 mg to about 600 mg of the compound of formula (I) and from about 50 mg to about 200 mg of benzbromarone is administered.

In some embodiments, from about 50 mg to about 600 mg of the compound of formula (I) is administered. In further or additional embodiments, from about 100 mg to about 400 mg of the compound of formula (I) is administered. Generally, any of the compounds administered for the treatment of any of the disorders or in any of the therapies described herein are administered in a therapeutically effective amount of the compound or compounds, either alone or in combination. It is to be understood that therapeutically effective amounts may be lowered in combination therapies than in mono-therapies. In further or additional embodiments, a subject treated according to any method described herein has a disorder characterized by an abnormally high content of uric acid in one or more tissues or organs of the subject. In further or additional embodiments, the disorder is characterized by overproduction of uric acid, low excretion of uric acid, tumor lysis, a blood disorder or a combination thereof. In further or additional embodiments, the blood disorder is polycythemia or myeloid metaplasia. In further or additional embodiments, the subject in need of decreased serum uric acid levels and/or in need of any therapy described herein is suffering from gout, a recurrent gout attack, gouty arthritis, hyperuricaemia, hypertension, a cardiovascular disease, coronary heart disease, Lesch-Nyhan syndrome, Kelley-Seegmiller syndrome, kidney disease, kidney stones, kidney failure, joint inflammation, arthritis, urolithiasis, plumbism, hyperparathyroidism, psoriasis or sarcoidosis. In further or additional embodiments, following a therapy described herein the uric acid levels of an individual receiving such a therapy are decreased by at least about 10% (or >10%). In further or additional embodiments, the uric acid levels are decreased by at least about 25% (or >25%). In further or additional embodiments, the uric acid levels are decreased by at least about 50% (or >50%). In further or additional embodiments, the tissue or organ is blood.

In further or additional embodiments, following a therapy described herein the blood uric acid level of an individual receiving such a therapy is decreased by at least about 1 mg/dL. In further or additional embodiments, the blood uric acid level is decreased by at least about 1.5 mg/dL. In further or additional embodiments, the blood uric acid level is decreased by at least about 2 mg/dL. In further or additional embodiments, the blood uric acid level is decreased by at least about 2.5 mg/dL. In further or additional embodiments, the blood uric acid level is decreased by at least about 3 mg/dL. In further or additional embodiments, the blood uric acid level is decreased by at least about 3.5 mg/dL. In further or additional embodiments, the blood uric acid level is decreased by at least about 4 mg/dL. In further or additional embodiments, the blood uric acid level is decreased by at least about 4.5 mg/dL. In further or additional embodiments, the blood uric acid level is decreased by at least about 5 mg/dL. In further or additional embodiments, the blood uric acid level is decreased by at least about 5.5 mg/dL. In further or additional embodiments, the blood uric acid level is decreased by at least about 6 mg/dL. In further or additional embodiments, the blood uric acid level is decreased by more than about 6 mg/dL. As used herein, blood uric acid levels may refer to uric acid levels found in whole blood, or its component parts, such as serum. Conversely, disclosures of serum uric acid levels herein should be understood to describe disclosures of blood uric acid levels.

In further or additional embodiments, following a therapy described herein the blood uric acid level of an individual receiving such a therapy decreases to at least about 7 mg/dL (i.e., decreased to 7 mg/dL or less). In further or additional embodiments, the blood uric acid level decreases to at least about 6.5 mg/dL. In further or additional embodiments, the blood uric acid level decreases to at least about 6 mg/dL. In further or additional embodiments, the blood uric acid level decreases to at least about 5.5 mg/dL. In further or additional embodiments, the blood uric acid level decreases to at least about 5 mg/dL. In further or additional embodiments, the blood uric acid level decreases to at least about 4.5 mg/dL. In further or additional embodiments, the blood uric acid level decreases to at least about 4 mg/dL.

Also provided herein are methods for treating hypoxanthine-guanine phosphoribosyltransferase (HPRT) deficiency in a subject, the HPRT being refractory, non-responsive, and/or resistant to allopurinol, febuxostat, probenecid, tranilast, sulfinpyrazone, losartan, fenofibrate, benzbromarone and/or PNP-inhibitor monotherapy, comprising administering a therapeutically effective amount of a compound of formula (I). In further or alternative embodiments, provided herein are methods for treating hypoxanthine-guanine phosphoribosyltransferase (HPRT) deficiency in a subject, comprising administering to the subject allopurinol, febuxostat, probenecid, tranilast, sulfinpyrazone, losartan, fenofibrate, benzbromarone or a PNP-inhibitor, and a compound of formula (I). In some embodiments, allopurinol or febuxostat is administered. In further or additional embodiments, allopurinol is administered. In further or additional embodiments, febuxostat is administered. In further or additional embodiments, M is Na or H. In further or additional embodiments, M is Na. In further or additional embodiments, M is H. In further or additional embodiments, allopurinol is administered and M is Na. In further or additional embodiments, allopurinol is administered and M is H. In further or additional embodiments, febuxostat is administered and M is Na. In further or additional embodiments, febuxostat is administered and M is H.

Also provided herein are methods for decreasing uric acid levels in one or more tissues or organs of a subject, comprising administering to the subject allopurinol, febuxostat, probenecid, tranilast, sulfinpyrazone, losartan, fenofibrate, benzbromarone or a PNP-inhibitor, and a compound of formula (I), and wherein the reduction in uric acid levels results in a reduction in hypertension or cardiovascular events. In some embodiments, allopurinol or febuxostat is administered. In further or additional embodiments, allopurinol is administered. In further or additional embodiments, febuxostat is administered. In further or additional embodiments, M is Na or H. In further or additional embodiments, M is Na. In further or additional embodiments, allopurinol is administered and M is Na. In further or additional embodiments, allopurinol is administered and M is H. In further or additional embodiments, febuxostat is administered and M is Na. In further or additional embodiments, febuxostat is administered and M is H.

Also provided herein are methods for preventing, slowing, or arresting the formation of or reducing the size of tophi/tophus in a subject, comprising administering to the subject allopurinol and a compound of formula (I). In some embodiments, M is Na or H. In further or additional embodiments, M is H.

Also provided herein are methods for increasing the velocity of tophi size reduction in a subject, comprising administering to the subject allopurinol and a compound of formula (I). In some embodiments, M is Na or H. In further or additional embodiments, M is H.

Also provided herein are methods for preventing, slowing, or arresting the formation of or reducing the size of tophi/tophus in a subject, comprising administering to the subject febuxostat and a compound of formula (I). In some embodiments, M is Na or H. In further or additional embodiments, M is H.

Also provided herein are methods for increasing the velocity of tophi size reduction in a subject, comprising administering to the subject febuxostat and a compound of formula (I). In some embodiments, M is Na or H. In further or additional embodiments, M is H.

Also provided herein are methods for increasing the velocity of tophi size reduction in a subject, comprising administering to the subject allopurinol, febuxostat, probenecid, tranilast, sulfinpyrazone, losartan, fenofibrate, benzbromarone or a PNP-inhibitor and a compound of formula (I). In some embodiments, M is Na or H. In further or additional embodiments, M is H.

Also described herein are methods of reducing serum uric acid levels in a subject, comprising administering to the subject a compound of formula (I)

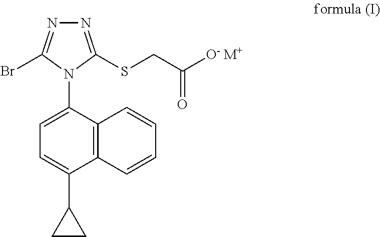

wherein M is H, Na, Ca, Mg, Zn, K, Al, piperazine or meglumine and wherein prior to administration the subject has a serum uric acid level greater than about 6.0 mg/dL and wherein after administration the subject has a serum uric acid level that is reduced and is less than about 6.0 mg/dL and wherein the subject has a creatinine clearance rate below about 60 mL/minute. In some embodiments, the subject has a creatinine clearance rate of from about 30 mL/minute to about 60 mL/minute. In some embodiments, the subject has a creatinine clearance rate below about 30 mL/minute. In various embodiments, prior to treatment, such subjects have a serum uric acid level of greater than 6.5 mg/dL, greater than 7.0 mg/dL, greater than 7.5 mg/dL, greater than 8.0 mg/dL, or more.

Also provided herein are methods of reducing serum uric acid levels in a subject suffering from hyperuricemia, comprising administering to the subject a compound of formula (I), wherein the subject has a creatinine clearance rate below about 60 mL/minute and wherein after administration the subject has a serum uric acid level less than about 6.0 mg/dL. In some embodiments, the subject has a creatinine clearance rate of from about 30 mL/minute to about 60 mL/minute. In some embodiments, the subject has a creatinine clearance rate below about 30 mL/minute. In various embodiments, prior to treatment, such subjects have a serum uric acid level of greater than 6.5 mg/dL, greater than 7.0 mg/dL, greater than 7.5 mg/dL, greater than 8.0 mg/dL, or more. In some embodiments, the method further comprises administering allopurinol or febuxostat.

Provided in certain embodiments herein is a method of reducing serum uric acid levels in a subject with renal impairment (e.g., mild or moderate renal impairment), the method comprising administering to the subject a compound of formula (I). In specific embodiments, the method reduces the level of serum uric acid levels in the subject. In certain embodiments, elevated levels of serum uric acid include amounts of greater than 6.0 mg/dL, greater than 6.5 mg/dL, greater than 7.0 mg/dL, greater than 7.5 mg/dL, greater than 8.0 mg/dL, or more. In some embodiments, following administration of a compound of formula (I) according to any method described herein, serum uric acid levels are reduced to less than 6.5 mg/dL, less than 6.0 mg/dL, less than 5.5 mg/dL, less than 5.0 mg/dL, or less.

Provided in some embodiments herein is a method of treating gout in a subject with renal impairment (e.g., mild or moderate renal impairment), the method comprising administering to the subject a compound of formula (I). Also in certain embodiments herein is a method of treating hyperuricemia in a subject with renal impairment (e.g., mild or moderate renal impairment), the method comprising administering to the subject a compound of formula (I).

In various embodiments, renal impairment may be determined in any suitable manner. In some embodiments, the subject having renal impairment has a creatinine clearance rate of less than 80 mL/min. In more specific embodiments, the subject having renal impairment has a creatinine clearance rate of less than 60 mL/min. In still more specific embodiments, the subject having renal impairment has a creatinine clearance rate of less than 50 mL/min. In yet more specific embodiments, the subject having renal impairment has a creatinine clearance rate of less than 40 mL/min. In more specific embodiments, the subject having renal impairment has a creatinine clearance rate of less than 30 mL/min. In more specific embodiments, the subject having renal impairment has a creatinine clearance rate of between 30 mL/min and 60 mL/min.

In various embodiments, a compound of formula (I) is administered in a method described herein in any suitable amount. In some embodiments, from about 50 mg to about 600 mg of the compound of formula (I) is administered. In further or additional embodiments, from about 100 mg to about 400 mg of the compound of formula (I) is administered. In some embodiments, the compound of formula (I) is administered once daily. In further or additional embodiments, the compound of formula (I) is administered more than once daily. In further or additional embodiments, the compound of formula (I) is administered twice daily.

In some embodiments, M is H or Na. In further or additional embodiments, M is Na. In some embodiments the compound of formula (I) is sodium 2-(5-bromo-4-(4-cyclopropylnaphthalen-1-yl)-4H-1,2,4-triazol-3-ylthio)acetate.

In some embodiments, the subject has a disorder characterized by an abnormally high content of uric acid in one or more tissues, joints, organs or blood of the subject. In further or additional embodiments, the disorder is characterized by overproduction of uric acid, low excretion of uric acid, tumor lysis, a blood disorder or a combination thereof. In further or additional embodiments, the blood disorder is polycythemia or myeloid metaplasia In some embodiments, the subject is suffering from gout, a recurrent gout attack, gouty arthritis, hyperuricaemia, hypertension, a cardiovascular disease, coronary heart disease, Lesch-Nyhan syndrome, Kelley-Seegmiller syndrome, kidney disease, kidney stones, kidney failure, joint inflammation, arthritis, urolithiasis, plumbism, hyperparathyroidism, psoriasis or sarcoidosis In further or additional embodiments, the subject is suffering from gout In further or additional embodiments, the subject is suffering from joint inflammation. In further or additional embodiments, the joint inflammation is caused by deposits of uric acid crystals in the joint. In further or additional embodiments, the uric acid crystals are deposited in the joint fluid (synovial fluid) or joint lining (synovial lining).

In some embodiments, after administration the subject has a serum uric acid level less than about 6.5 mg/dL. In further or additional embodiments, after administration the subject has a serum uric acid level less than about 6 mg/dL. In further or additional embodiments, after administration the subject has a serum uric acid level less than about 5 mg/dL. In further or additional embodiments, after administration the subject has a serum uric acid level less than about 4.5 mg/dL. In further or additional embodiments, after administration the subject has a serum uric acid level less than about 4 mg/dL.

Further provided herein is a method of treating or preventing hyperuricemia or gout in a subject, comprising administering to the subject (i) allopurinol, or febuxostat, or a combination thereof, and (ii) a compound of formula (I), wherein M is H, Na, Ca, Mg, Zn, K, Al, piperazine, or meglumine. In some embodiments, M is H. In other embodiments, M is Na. In certain embodiments, from about 100 mg to about 400 mg of the compound of formula (I) is administered.

In some embodiments, the gout or hyperuricemia is refractory, non-responsive, or resistant to allpurinol monotherapy, febuxostat monotherapy, PNP-inhibitor monotherapy, probenecid monotherapy, tranilast monotherapy, sulfinpyrazone monotherapy, losartan monotherapy, fenofibrate monotherapy, and/or benzbromarone monotherapy.

In certain embodiments, the method of treating or preventing hyperuricemia or gout comprises administering from about 100 mg to about 1000 mg of allopurinol and a compound of formula (I). In some embodiments, the subject has received treatment with allopurinol prior to administration and the allopurinol treatment does not decrease serum uric acid levels below about 6 mg/dL, and after administration of allopurinol and a compound of formula (I), serum uric acid levels decrease below about 6 mg/dL.

In some embodiments, the method of treating or preventing hyperuricemia or gout comprises administering from about 20 mg to about 150 mg of febuxostat and a compound of formula (I). In some embodiments, the subject has received treatment with febuxostat and the febuxostat treatment does not decrease serum uric acid levels below about 6 mg/dL, and after administration of febuxostat and a compound of formula (I), serum uric acid levels decrease below about 6 mg/dL.

Also provided herein is a pharmaceutical composition comprising (i) a compounds selected from the group consisting of allopurinol, febuxostat, a PNP-inhibitor (e.g., BCX4208), probenecid, tranilast, sulfinpyrazone, losartan, fenofibrate, benzbromarone, and a combination thereof, (ii) a compound of formula (I), and (iii) at least one pharmaceutically acceptable carrier.

In specific embodiments, the pharmaceutical composition comprises (i) allopurinol, (ii) a compound of formula (I), and (iii) at least one pharmaceutically acceptable carrier. In some embodiments, the compound of formula (I) is 2-(5-bromo-4-(4-cyclopropylnaphthalen-1-yl)-4H-1,2,4-triazol-3-ylthio)acetic acid. In other embodiments, the compound of formula (I) is sodium 2-(5-bromo-4-(4-cyclopropylnaphthalen-1-yl)-4H-1,2,4-triazol-3-ylthio)acetate. In some embodiments, the composition comprises about 100 mg to about 1000 mg of a compound of formula (I). In certain embodiments, the composition comprises about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg of a compound of formula (I). In other embodiments, the composition comprises about 100 mg to about 400 mg of a compound of formula (I). In some embodiments, the composition comprises from about 100 mg to about 1000 mg of allopurinol. In certain embodiments, the composition comprises about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg of allopurinol.

In further specific embodiments, the pharmaceutical composition comprises (i) febuxostat, (ii) a compound of formula (I), and (iii) at least one pharmaceutically acceptable carrier. In some embodiments, the compound of formula (I) is 2-(5-bromo-4-(4-cyclopropylnaphthalen-1-yl)-4H-1,2,4-triazol-3-ylthio)acetic acid. In other embodiments, the compound of formula (I) is sodium 2-(5-bromo-4-(4-cyclopropylnaphthalen-1-yl)-4H-1,2,4-triazol-3-ylthio)acetate. In some embodiments, the composition comprises about 50 mg to about 1000 mg of a compound of formula (I). In certain embodiments, the composition comprises about 50 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg of a compound of formula (I). In other embodiments, the composition comprises about 100 mg to about 400 mg of a compound of formula (I). In some embodiments, the composition comprises from about 20 mg to about 200 mg of febuxostat. In certain embodiments, the composition comprises about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, or about 200 mg of febuxostat.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 represents a study design diagram for the events described in Example 1.

FIG. 2 represents a scheme describing the oral administration of Febuxostat or drug 1 (200 mg)/placebo in week one; Febuxostat and drug 1 (200 mg)/placebo in week two, and Febuxostat or drug 1 (200 mg)/placebo in week three, in 2 randomized sequences, to healthy subjects, according to the protocol described in Example 10.

FIG. 3 represents a graph of % mean serum uric acid changes from baseline after administration of drug 1 (200 mg), placebo and/or Febuxostat during weeks 1 & 2, as described in Example 11.

FIG. 4 represents a graph of % mean serum uric acid changes from baseline after administration of drug 1 (200 mg), placebo and/or Febuxostat during weeks 1, 2 & 3, as described in Example 12.

FIG. 5 represents a graph of creatinine clearance (CrCL) versus % change in serum uric acid levels on day 14, after 14 days dosing drug 1, 400 mg qd, as described in Example 20.

FIG. 6 represents a graph of creatinine clearance (CrCL; MDRD method) versus % change in serum uric acid levels on day 14, after 14 days dosing drug 1, 400 mg qd, as described in Example 20.

FIG. 7 represents a study design diagram for evaluation of interactions between drug 1 and allopurinol, as described in Example 21.

FIG. 8 represents a graph of mean serum uric acid levels following allopurinol and drug 1 monotherapy and combination over 3 weeks in Gout Patients with Hyperuricemia, as described in Example 22.

FIG. 9 represents a study design diagram for evaluation of interactions between drug 1 and febuxostat, as described in Example 23.

FIG. 10 represents a graph of mean serum uric acid levels following febuxostat monotherapy and combination with drug 1 over 3 weeks in Gout Patients with Hyperuricemia, as described in Example 24.

FIG. 11 represents a graph of % serum uric acid change from baseline following febuxostat monotherapy and combination with drug 1 over 3 weeks in Gout Patients with Hyperuricemia, as described in Example 24.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

In various embodiments provided herein are methods (such as those set forth in the Summary) comprising the administration to an individual a compound having the following formula:

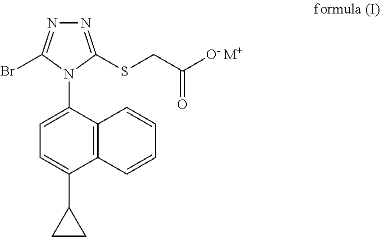

wherein M is H, Na, Ca, Mg, Zn, K, Al, piperazine or meglumine

### Uric Acid

In certain instances, purines (i.e., adenine, guanine), derived from food or tissue turnover (cellular nucleotides undergo continuous turnover), are catabolized in humans to their final oxidation product, uric acid. In certain instances, guanine is oxidized to xanthine, which is turn is further oxidized to uric acid by the action of xanthine oxidase; adenosine is converted to inosine which is further oxidized to hypoxanthine. In certain instances, xanthine oxidase oxidizes hypoxanthine to xanthine, and further to uric acid. In certain instances, as part of the reverse process, the enzyme hypoxanthine-guanine phosphoribosyltransferase (HGPRT) salvages guanine and hypoxanthine.

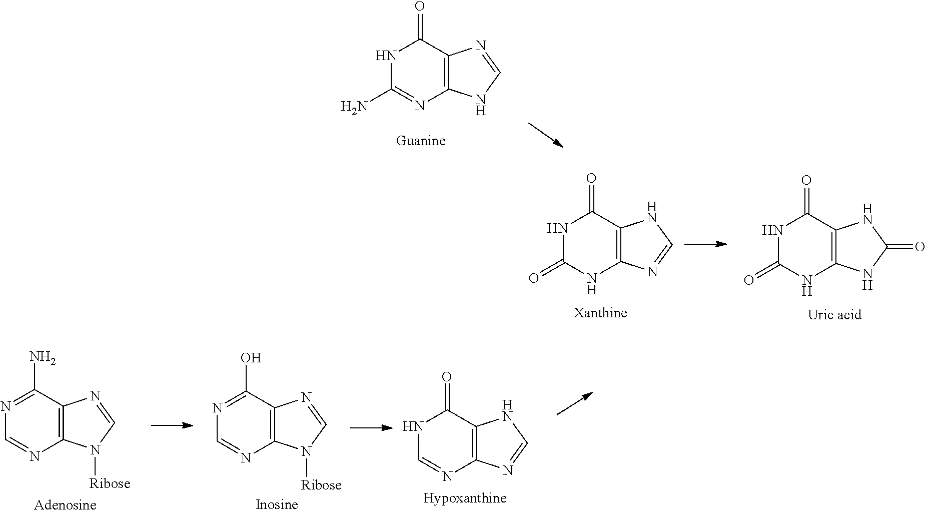

In certain instances, the keto form of uric acid is in equilibrium with the enol form which loses a proton at physiological pH to form urate. In certain instances, (e.g., under serum conditions (pH 7.40, 37° C.)), about 98% of uric acid is ionized as the monosodium urate salt. In certain instances, urate is a strong reducing agent and potent antioxidant. In humans, about half the antioxidant capacity of plasma comes from uric acid. As used herein, concentrates of uric acid are understood to include all forms of uric acid, including the enol form and urate.

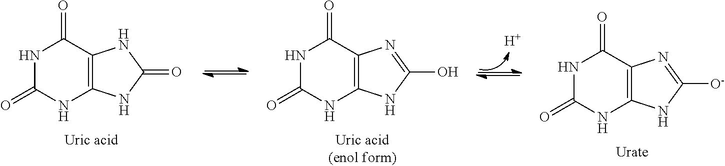

In certain instances, most uric acid dissolves in blood and passes to the kidneys, where it is excreted by glomerular filtration and tubular secretion. In certain instances, a substantial fraction of uric acid is reabsorbed by the renal tubules. One of the peculiar characteristics of the uric acid transport system is that, although the net activity of tubular function is reabsorption of uric acid, the molecule is both secreted and reabsorbed during its passage through the nephron. In certain instances, reabsorption dominates in the S1 and S3 segments of the proximal tubule and secretion dominates in the S2 segment. In certain instances, the bidirectional transport results in drugs that inhibit uric acid transport decreasing, rather than increasing, the excretion of uric acid, compromising their therapeutic usefulness. In certain instances, normal uric acid levels in human adults (5.1+/−0.93 mg/dL) are close to the limits of urate solubility (˜7 mg/dL at 37° C.), which creates a delicate physiologic urate balance. In certain instances, the normal uric acid range for females is approximately 1 mg/dL below the male range.

### Hyperuricemia

In certain instances, hyperuricemia is characterized by higher than normal blood levels of uric acid, sustained over long periods of time. In certain instances, increased blood urate levels may be due to enhanced uric acid production (˜10-20%) and/or reduced renal excretion (˜80-90%) of uric acid. In certain instances, causes of hyperuricemia may include:
Obesity/weight gain
Excessive alcohol use
Excessive dietary purine intake (foods such as shellfish, fish roe, scallops, peas lentils, beans and red meat, particularly offal—brains, kidneys, tripe, liver)
Certain medications, including low-dose aspirin, diuretics, niacin, cyclosporine, pyrazinamide, ethambutol, some high blood pressure drugs and some cancer chemotherapeutics, immunosuppressive and cytotoxic agents
Specific disease states, particularly those associated with a high cell turnover rate (such as malignancy, leukemia, lymphoma or psoriasis), and also including high blood pressure, hemoglobin diseases, hemolytic anemia, sickle cell anemia, various nephropathies, myeloproliferative and lymphoproliferative diseases, hyperparathyroidism, renal disease, conditions associated with insulin resistance and diabetes mellitus, and in transplant recipients, and possibly heart disease
Inherited enzyme defects
Abnormal kidney function (e.g. increased ATP turn over, reduced glomerular urate filtration)
Exposure to lead (plumbism or “saturnine gout”)

In certain instances, hyperuricemia may be asymptomatic, though is associated with the following conditions: gout, gouty arthritis, uric acid stones in the urinary tract (urolithiasis), deposits of uric acid in the soft tissue (tophi), deposits of uric acid in the kidneys (uric acid nephropathy), and impaired kidney function, possibly leading to chronic and acute renal failure.

### Gout

### Prevalence

Gout is a condition that results from uric acid crystals depositing in tissues of the body. It is often related to an inherited abnormality in the body's ability to process uric acid, but may also be exacerbated by a diet high in purines. Defective uric acid processing may lead to elevated levels of uric acid in the blood causing recurring attacks of joint inflammation (arthritis), uric acid deposits in and around the joints, decreased kidney function, and kidney stones. Approximately 3-5 million people in the United States suffer from attacks of gout with attacks 6 to 9 times more common in men than in women (see Sanders and Wortmann, “Harrison's Principles of Internal Medicine”, 16th Edition; 2005; Food and Drug Administration (FDA) Advisory Committee Meeting, Terkeltaub presentation, June 2004; Terkeltaub, “Gout”, N Engl J Med., 349, 1647-55, 2003).

In certain instances, gout is one of the most common forms of arthritis, accounting for approximately 5% of all arthritis cases. In certain instances, kidney failure and urolithiasis occur in 10-18% of individuals with gout and are common sources of morbidity and mortality from the disease.

### Tophi and Tophaceous Gout

One study (Perez-Ruiz et al., Arthritis & Rheumatism (Arthritis Care & Research), 2002, 47, (4), 356-360) of 63 patients with crystal-confirmed tophaceous gout, examined the relationship between serum urate levels during therapy and the velocity of reduction of tophi in patients with chronic tophaceous gout. It was observed that the velocity of tophi reduction was linearly related to the mean serum urate level during therapy; the lower the serum urate level achieved during urate-lowering therapy (ULT), the faster the reduction in tophaceous deposits.

### Leading Causes

In most cases, gout is associated with hyperuricemia. In certain instances, individuals suffering from gout excrete approximately 40% less uric acid than nongouty individuals for any given plasma urate concentration. In certain instances, urate levels increase until the saturation point is reached. In certain instances, precipitation of urate crystals occurs when the saturation point is reached. In certain instances, these hardened, crystallized deposits (tophi) form in the joints and skin, causing joint inflammation (arthritis). In certain instances, deposits are be made in the joint fluid (synovial fluid) and/or joint lining (synovial lining). Common areas for these deposits are the large toe, feet, ankles and hands (less common areas include the ears and eyes). In certain instances, the skin around an affected joint becomes red and shiny with the affected area being tender and painful to touch. In certain instances, gout attacks increase in frequency. In certain instances, untreated acute gout attacks lead to permanent joint damage and disability. In certain instances, tissue deposition of urate leads to: acute inflammatory arthritis, chronic arthritis, deposition of urate crystals in renal parenchyma and urolithiasis. In certain instances, the incidence of gouty arthritis increases 5 fold in individuals with serum urate levels of 7 to 8.9 mg/dL and up to 50 fold in individuals with levels >9 mg/dL (530 μmol/L). In certain instances, individuals with gout develop renal insufficiency and end stage renal disease (i.e., “gouty nephropathy”). In certain instances, gouty nephropathy is characterized by a chronic interstitial nephropathy, which is promoted by medullary deposition of monosodium urate.

In certain instances, gout includes painful attacks of acute, monarticular, inflammatory arthritis, deposition of urate crystals in joints, deposition of urate crystals in renal parenchyma, urolithiasis (formation of calculus in the urinary tract), and nephrolithiasis (formation of kidney stones). In certain instances, secondary gout occurs in individuals with cancer, particularly leukemia, and those with other blood diseases (e.g. polycythemia, myeloid metaplasia, etc).

### Symptoms

In certain instances, attacks of gout develop very quickly, frequently the first attack occurring at night. In certain instances, symptoms include sudden, severe joint pain and extreme tenderness in the joint area, joint swelling and shiny red or purple skin around the joint. In certain instances, the attacks are infrequent lasting 5-10 days, with no symptoms between episodes. In certain instances, attacks become more frequent and last longer, especially if the disease is not controlled. In certain instances, episodes damage the affected joint(s) resulting in stiffness, swelling, limited motion and/or persistent mild to moderate pain.

### Treatment

In certain instances, gout is treated by lowering the production of uric acid. In certain instances, gout is treated by increasing the excretion of uric acid. In certain instances, gout is treated by URAT 1, xanthine oxidase, xanthine dehydrogenase, xanthine oxidoreductase, a purine nucleoside phosphorylase (PNP) inhibitor, a uric acid transporter (URAT) inhibitor, a glucose transporter (GLUT) inhibitor, a GLUT-9 inhibitor, a solute carrier family 2 (facilitated glucose transporter), member 9 (SLC2A9) inhibitor, an organic anion transporter (OAT) inhibitor, an OAT-4 inhibitor, or combinations thereof. In general, the goals of gout treatment are to i) reduce the pain, swelling and duration of an acute attack, and ii) prevent future attacks and joint damage. In certain instances, gout attacks are treated successfully using a combination of treatments. In certain instances, gout is one of the most treatable forms of arthritis.

i) Treating the Gout Attack.

In certain instances, the pain and swelling associated with an acute attack of gout can be addressed with medications such as acetaminophen, steroids, nonsteroidal anti-inflammatory drugs (NSAIDs), adrenocorticotropic hormone (ACTH) or colchicine. In certain instances, proper medication controls gout within 12 to 24 hours and treatment is stopped after a few days. In certain instances, medication is used in conjunction with rest, increased fluid intake, ice-packs, elevation and/or protection of the affected area/s. In certain instances, the aforementioned treatments do not prevent recurrent attacks and they do not affect the underlying diseases of abnormal uric acid metabolism.

ii) Preventing Future Attacks.

In certain instances, reducing serum uric acid levels below the saturation level is the goal for preventing further gout attacks. In some cases, this is achieved by decreasing uric acid production (e.g. allopurinol), or increasing uric acid excretion with uricosuric agents (e.g. probenecid, sulfinpyrazone, benzbromarone).

In certain instances, allopurinol inhibits uric acid formation, resulting in a reduction in both the serum and urinary uric acid levels and becomes fully effective after 2 to 3 months.

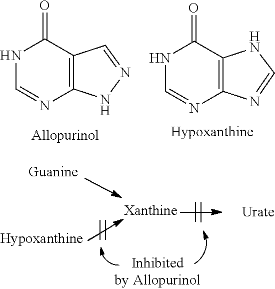

Allopurinol is a structural analogue of hypoxanthine, (differing only in the transposition of the carbon and nitrogen atoms at positions 7 and 8), which in certain instances, inhibits the action of xanthine oxidase, the enzyme responsible for the conversion of hypoxanthine to xanthine, and xanthine to uric acid. In certain instances, it is metabolized to the corresponding xanthine analogue, alloxanthine (oxypurinol), which is also an inhibitor of xanthine oxidase. In certain instances, alloxanthine, though more potent in inhibiting xanthine oxidase, is less pharmaceutically acceptable due to low oral bioavailability. In certain instances, fatal reactions due to hypersensitivity, bone marrow suppression, hepatitis, and vasculitis have been reported with Allopurinol. In certain instances, the incidence of side effects may total 20% of all individuals treated with the drug. Treatment for diseases of uric acid metabolism has not evolved significantly in the following two decades since the introduction of allopurinol.

In certain instances, uricosuric agents (e.g., probenecid, sulfinpyrazone, and benzbromarone) increase uric acid excretion. In certain instances, probenecid causes an increase in uric acid secretion by the renal tubules and, when used chronically, mobilizes body stores of urate. In certain instances, 25-50% of individuals treated with probenecid fail to achieve reduction of serum uric acid levels <6 mg/dL. In certain instances, insensitivity to probenecid results from drug intolerance, concomitant salicylate ingestion, and renal impairment. In certain instances, one-third of the individuals develop intolerance to probenecid. In certain instances, administration of uricosuric agents also results in urinary calculus, gastrointestinal obstruction, jaundice and anemia.

### Plumbism or “Saturnine Gout”

In certain instances, excessive exposure to lead (lead poisoning or plumbism) results in “saturnine gout,” a lead-induced hyperuricemia that results from lead inhibition of tubular urate transport causing decreased renal excretion of uric acid. In certain instances, more than 50% of individuals suffering from lead nephropathy suffer from gout. In certain instances, acute attacks of saturnine gout occur in the knee more frequently than the big toe. In certain instances, renal disease is more frequent and more severe in saturnine gout than in primary gout. In certain instances, treatment consists of excluding the individual from further exposure to lead, the use of chelating agents to remove lead, and control of acute gouty arthritis and hyperuricaemia. In certain instances, saturnine gout is characterized by less frequent attacks than primary gout. In certain instances, lead-associated gout occurs in pre-menopausal women, an uncommon occurrence in non lead-associated gout.

### Lesch-Nyhan Syndrome

In certain instances, Lesch-Nyhan syndrome (LNS or Nyhan's syndrome) affects about one in 100,000 live births. In certain instances, LNS is caused by a genetic deficiency of the enzyme hypoxanthine-guanine phosphoribosyltransferase (HGPRT). In certain instances, LNS is an X-linked recessive disease. In certain instances, LNS is present at birth in baby boys. In certain instances, the disease leads to severe gout, poor muscle control, and moderate mental retardation, which appear in the first year of life. In certain instances, the disease also results in self-mutilating behaviors (e.g., lip and finger biting, head banging) beginning in the second year of life. In certain instances, the disease also results in gout-like swelling in the joints and severe kidney problems. In certain instances, the disease leads neurological symptoms include facial grimacing, involuntary writhing, and repetitive movements of the arms and legs similar to those seen in Huntington's disease. The prognosis for individuals with LNS is poor. In certain instances, the life expectancy of an untreated individual with LNS is less than about 5 years. In certain instances, the life expectancy of a treated individual with LNS is greater than about 40 years of age.

### Hyperuricemia and Other Diseases

In certain instances, hyperuricemia is found in individuals with cardiovascular disease (CVD) and/or renal disease. In certain instances, hyperuricemia is found in individuals with prehypertension, hypertension, increased proximal sodium reabsorption, microalbuminuria, proteinuria, kidney disease, obesity, hypertriglyceridemia, low high-density lipoprotein cholesterol, hyperinsulinemia, hyperleptinemia, hypoadiponectinemia, peripheral, carotid and coronary artery disease, atherosclerosis, congestive heart failure, stroke, tumor lysis syndrome, endothelial dysfunction, oxidative stress, elevated renin levels, elevated endothelin levels, and/or elevated C-reactive protein levels. In certain instances, hyperuricemia is found in individuals with obesity (e.g., central obesity), high blood pressure, hyperlipidemia, and/or impaired fasting glucose. In certain instances, hyperuricemia is found in individuals with metabolic syndrome. In certain instances, gouty arthritis is indicative of an increased risk of acute myocardial infarction. In some embodiments, administration of a compound described herein to an individual are useful for decreasing the likelihood of a clinical event associated with a disease or condition linked to hyperuricemia, including, but not limited to, prehypertension, hypertension, increased proximal sodium reabsorption, microalbuminuria, proteinuria, kidney disease, obesity, hypertriglyceridemia, low high-density lipoprotein cholesterol, hyperinsulinemia, hyperleptinemia, hypoadiponectinemia, peripheral, carotid and coronary artery disease, atherosclerosis, congestive heart failure, stroke, tumor lysis syndrome, endothelial dysfunction, oxidative stress, elevated renin levels, elevated endothelin levels, and/or elevated C-reactive protein levels.

In some embodiments, a compound described herein is administered to an individual suffering from a disease or condition requiring treatment with a diuretic. In some embodiments, a compound described herein are administered to an individual suffering from a disease or condition requiring treatment with a diuretic, wherein the diuretic causes renal retention of urate. In some embodiments, the disease or condition is congestive heart to failure or essential hypertension.

In some embodiments, administration of a compound described herein to an individual is useful for improving motility or improving quality of life.

In some embodiments, administration of a compound described herein to an individual is useful for treating or decreasing the side effects of cancer treatment.

In some embodiments, administration of a compound described herein to an individual is useful for decreasing kidney toxicity of cis-platin.

### Gout Treatment

Successful treatment aims to reduce both the pain associated with acute gout flare and long-term damage to the affected joints (Emerson, “The Management of Gout”, N Engl J Med., 334(7), 445-451, 1996). Therapeutic goals include providing rapid and safe pain relief, preventing further attacks, preventing the formation of tophi and subsequent arthritis, and avoiding exacerbating other medical conditions. Initiation of treatment depends upon the underlying causes of hyperuricemia, such as renal function, diet, and medications. While gout is a treatable condition, there are limited treatments available for managing acute and chronic gout and a number of adverse effects are associated with current therapies. Medication treatment of gout includes pain management, prevention or decrease in joint inflammation during an acute gouty attack, and chronic long-term therapy to maintain decreased serum uric acid levels.

Nonsteroidal anti-inflammatory drugs (NSAIDs) are effective anti-inflammatory medications for acute gout but are frequently associated with irritation of the gastrointestinal (GI) system, ulceration of the stomach and intestines, and occasionally intestinal bleeding (Schlesinger, “Management of Acute and Chronic Gouty Arthritis Present State-of-the-Art”; Medications; 64 (21), 2399-2416, 2004; Pascual and Sivera, “Therapeutic advances in gout”; Curr Opin Rheumatol., Mar; 19(2), 122-7, 2007). Colchicine for acute gout is most commonly administered orally as tablets (every 1-2 hours until there is significant improvement in pain or the patient develops GI side effects such as severe diarrhea, nausea and vomiting), or intravenously. Corticosteroids, given in short courses, can be administered orally or injected directly into the inflamed joint.

Medications are available for reducing blood uric acid levels that either increase renal excretion of uric acid by inhibiting re-uptake or reduce production of uric acid by blockade of xanthine oxidase. These medicines are generally not initiated until after the inflammation from acute gouty arthritis has subsided because they may intensify the attack. If they are already being taken prior to the attack, they are continued and only adjusted after the attack has resolved. Since many subjects with elevated blood uric acid levels may not develop gouty attacks or kidney stones, the decision for prolonged treatment with uric acid-lowering medications is individualized.

### 1H-pyrazolo[3,4-d]pyrimidin-4(2H)-one (Allopurinol)

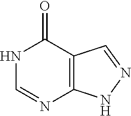

1H-pyrazolo[3,4-d]pyrimidin-4(2H)-one, or allopurinol, inhibits the synthesis of uric acid. Allopurinol has been marketed in the United States since 1964 as Zyloprim®. Other brand names include Allohexyl®, Allosig®, Progout®, Zyloric®, Lopurin® and Puricos®. Side effects of allopurinol, which can be severe, include, but are not limited to rash (occasionally life threatening toxic epidermal necrolysis), diarrhea, headache, fever, and platelet and white cell abnormalities.

### 2-(3-Cyano-4-isobutoxyphenyl)-4-methyl-1,3-thiazole-5-carboxylic acid (Febuxostat)

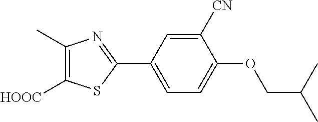 [SMILES: Cc1nc(-c2ccc(OCC(C)C)c(C#N)c2)sc1C(=O)O]

2-(3-Cyano-4-isobutoxyphenyl)-4-methyl-1,3-thiazole-5-carboxylic acid, or

Febuxostat, inhibits xanthine oxidase. Febuxostat was recently approved in the European Union (2008) and the United States (2009) and is marketed as Adenuric® and Uloric®. It blocks uric acid production and is most often used in subjects who overproduce uric acid and in older subjects for long-term gout treatment. It is often administered with NSAIDS or colchicine to reduce the possibility of a gout flare. Febuxostat has been associated with serious cardiovascular adverse events and liver function elevations in clinical trials.

### 4-(Dipropylsulfamoyl)benzoic acid (Probenacid) and 1,2-diphenyl-4-(2-(phenylsulfinyl)ethyl)pyrazolidine-3,5-dione (Sulfinpyrazone)

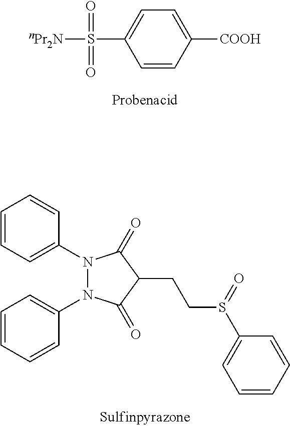

4-(Dipropylsulfamoyl)benzoic acid (Probenacid) and 1,2-diphenyl-4-(2-(phenylsulfinyl)ethyl)pyrazolidine-3,5-dione (Sulfinpyrazone) increase uric acid excretion into the urine (and therefore decrease uric acid blood levels). Since these medications may, in rare instances, cause kidney stones, they are avoided by subjects with a prior history and must be taken with sufficient fluid to promote the rapid elimination of uric acid from the urinary system to prevent stone formation.

### 2-{[(2E)-3-(3,4-dimethoxyphenyl)prop-2-enoyl]amino}benzoic acid (Tranilast)

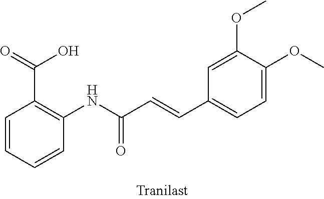 [SMILES: COc1ccc(/C=C/C(=O)Nc2ccccc2C(=O)O)cc1OC]

Tranilast an anti-inflammatory agent and it is being used for the treatment of asthma, allergic rhinitis, atopic dermatitis, and hypertrophic scarring. In some instances, tranilast has utility as an uricosuric agent while also demonstrating its anti-inflammatory activity.

### (1-((2′-(2H-tetrazol-5-yl)biphenyl-4-yl)methyl)-2-butyl-4-chloro-1H-imidazol-5-yl)methanol (Losartan) and

### isopropyl 2-(4-(4-chlorobenzoyl)phenoxy)-2-methylpropanoate (Fenofibrate)

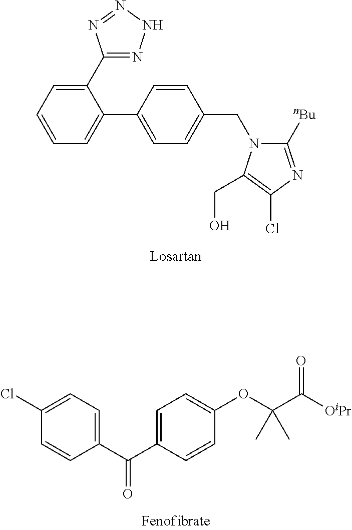

(1-((2′-(2H-tetrazol-5-yl)biphenyl-4-yl)methyl)-2-butyl-4-chloro-1H-imidazol-5-yl)methanol (Losartan) and isopropyl 2-(4-(4-chlorobenzoyl)phenoxy)-2-methylpropanoate (Fenofibrate) inhibit the URAT1 transporter (thus increasing renal elimination of uric acid). The uric acid lowering effects of losartan have been linked to some of its enhanced cardiovascular benefit compared to atenolol in the LIFE study (Hoieggen et al; “The impact of serum uric acid on cardiovascular outcomes in the LIFE study”; Kidney Int., 65, 1041-1049, 2003).

### (3,5-Dibromo-4-hydroxyphenyl)(2-ethylbenzofuran-3-yl)methanone (Benzbromarone)

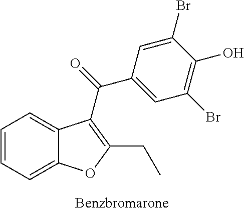 [SMILES: CCc1oc2ccccc2c1C(=O)c1cc(Br)c(O)c(Br)c1]

Benzbromarone is a uricosuric agent which blocks tubular reabsorption of uric acid. In certain instances, it has been used in the treatment of gout, especially when allopurinol fails or produces significant side effects.

### PNP-Inhibitors

Purine nucleoside phosphorylase (PNP) catalyzes the reversible phosphorolysis of purine ribonucleosides and 2′-deoxyribonucleosides to the free base and ribose-1-phosphate or 2′-deoxyribose-1-phosphate. PNP isolated from humans is specific for guanosine, inosine and certain analogs, although PNPs from other organisms show varying levels of specificity. Interest in PNP arises from its critical role in purine nucleoside metabolism and in T-cell function. Examples of PNP-inhibitors include but are not limited to 9-(3-Pyridylmethyl)-9-deazaguanine (BCX-34), 7-(((3R,4R)-3-hydroxy-4-(hydroxymethyl)pyrrolidin-1-yl)methyl)-3H-pyrrolo[3,2-d]pyrimidin-4(5H)-one (BCX4208), Forodesine (BCX-1777) and the like.

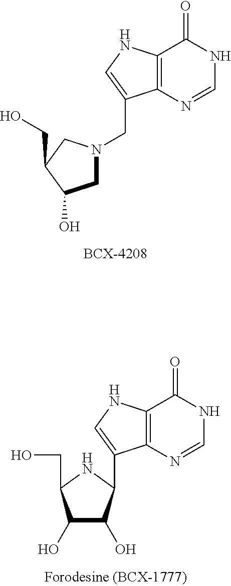

### Sodium 2-(5-bromo-4-(4-cyclopropylnaphthalen-1-yl)-4H-1,2,4-triazol-3-ylthio)acetate (“drug 1”) and

### 2-(5-bromo-4-(4-cyclopropylnaphthalen-1-yl)-4H-1,2,4-triazol-3-ylthio)acetic acid (“drug 2”)

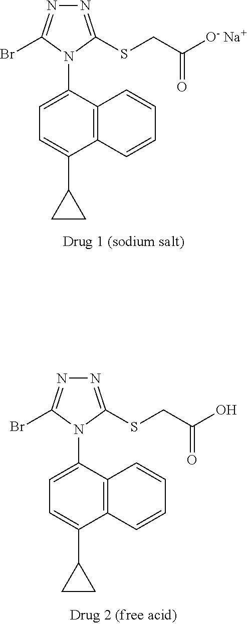

Sodium 2-(5-bromo-4-(4-cyclopropylnaphthalen-1-yl)-4H-1,2,4-triazol-3-ylthio)acetate is a uricosuric agent potentially useful for the treatment of gout (see WO/2009/070740 and WO 2010/028190). It displays uricosuric properties believed to act through inhibition of the uric acid transporter (URAT1) in the proximal tubule of the kidney, but does not significantly inhibit xanthine oxidase or PNP. In clinical studies, it was preferentially excreted through the kidney, reaching high concentrations in urine and exhibited a concentration-dependent inhibitory effect on the URAT1-mediated uptake of uric acid in vitro.

### Combination Therapy

Treatment of gout patients with a combination of allopurinol (200-300 mg per day) and a uricosuric agent (e.g., probenecid, benzbromarone) lowers sUA more effectively than allopurinol alone (Reinders et al, “Biochemical effectiveness of allopurinol and allopurinol/probenecid in previously benzbromarone-treated gout patients”, Clin Rheumatol, 26, 1459-1465, 2007). Studies in healthy volunteers treated with a combination of allopurinol and benzbromarone demonstrated a pharmacokinetic interaction whereby the plasma levels of oxypurinol, the metabolite of allopurinol, were reduced. This interaction ultimately impacted the pharmacodynamic effects of allopurinol as the sUA levels were not reduced as much as expected in these healthy volunteers (Colin et al, “Kinetics of Allopurinol and Oxipurinol After Chronic Oral Administration. Interaction with Benzbromarone”, Eur J Clin Pharmacol, 31,53-58, 1986). In contrast, gout patients treated with a combination of allopurinol and benzbromarone, display sUA levels reduced to a greater extent than gout patients treated with allopurinol alone (Müller, et al, “The Effect of Benzbromarone on Allopurinol/Oxypurinol Kinetics in Patients with Gout”, Eur J Clin Pharmacol, 44, 69-72, 1993). Additional benefits of combination therapy for gout patients include the potential for lower doses of one or both drugs thereby reducing the adverse effects of either drug used alone.

### EXAMPLES

The examples and preparations provided below further illustrate and exemplify the compounds of the present invention and methods of preparing such compounds. It is to be understood that the scope of the present invention is not limited in any way by the scope of the following examples and preparations.

### I. Clinical Trials

### Example 1

### Study Objectives

To compare the proportion of subjects whose sUA level is <6.0 mg/dL following 2 weeks of continuous treatment with drug 1 compared to allopurinol and placebo.
To evaluate the percent reduction from baseline in sUA levels following 2 weeks of continuous treatment with drug 1 in combination with allopurinol.
To evaluate the proportion of subjects whose sUA levels are <6.0 mg/dL, <5.0 mg/dL and <4.0 mg/dL at each visit.
To evaluate the absolute and percent reduction from baseline in sUA levels at each visit.
To evaluate the maximum percent reduction in sUA levels from baseline during the entire treatment period.
To evaluate percent change in 24-hour urine uric acid level from baseline to Day 15.
To evaluate the safety and tolerability of drug 1 in subjects with gout.
To evaluate the pharmacokinetics, safety and tolerability of drug 1 in combination with allopurinol in subjects with gout.

### Study Details

A randomized, double-blind, placebo-controlled, dose titration, safety and pharmacodynamics pilot study, in approximately 26 hyperuricemic subjects with symptomatic gout enrolled at 2-3 investigational sites in N America, of:

drug 1 versus placebo and open-label allopurinol (Cohort 1); and

drug 1 in combination with allopurinol versus continued allopurinol alone (Cohort 2)

The goal is to assess the pharmacodynamics and safety of drug 1 in establishing normal sUA concentrations in gout subjects with hyperuricemia.

Subjects are randomized 7 days prior to Day 1 in a double-blind fashion to receive drug 1, drug 1 matching placebo, or open-label allopurinol (Cohort 1) or drug 1 or drug 1 matching placebo in combination with allopurinol (Cohort 2).

To reduce the incidence of gout flares, colchicine 0.6 mg qd is administered to all subjects starting 14 days prior to the Baseline (Day 1) visit. Subjects continue colchicine administration throughout the Treatment Period, discontinuing at the End of Study visit (one week after the last dose of study medication).

Subjects in Cohort 2 dosed with allopurinol once daily 7 days prior to dosing with drug 1 (or placebo), continuing use of allopurinol until the End of Study visit (one week after the last dose of study medication).

24 hour urine samples are collected prior to dosing, starting on Day 1, to establish the baseline excretion of uric acid for each individual (except subjects randomized to allopurinol) in Cohort 1. Twenty-four hour urine samples are also collected starting on Day 8 just prior to dosing until the morning of Day 9, and starting on Day 14 just prior to dosing until the morning of Day 15. These samples are used to evaluate uric acid, allopurinol/oxypurinol (Cohort 2 only) and drug 1 excretion.

For subjects randomized to double-blind treatment, plasma samples for drug 1 concentration are collected to provide a general impression of systemic drug exposure in Cohort 1 and pharmacokinetics in combination with allopurinol in Cohort 2. Plasma samples are collected on Day 1 (1 hour and 8 hours post-dose), Day 8 (trough, 1 hour and 8 hours post-dose), Day 9 (24 hours after the Day 8 dose), Day 14 (trough and 8 hours post-dose), and Day 15 (24 hours after the Day 14 dose). For Cohort 2, an additional plasma sample is collected at pre-dose on Day 1 to assess allopurinol and oxypurinol plasma levels prior to starting double-blind treatment with drug 1 or placebo.

Subjects return for an End of Study visit approximately one week after the final dose of blinded study medication for sUA levels and safety assessments.

Cohort 1 approx 20 subjects randomized in 2:1:1 ratio treatment groups—drug 1:placebo:allopurinol.

Subjects randomized to double-blind treatment:
approx 10 subjects randomized to receive drug 1 200 mg qd (4 drug 1 50 mg capsules/day) for one week, followed by drug 1 400 mg qd (8 drug 1 50 mg capsules/day) for one week.
5 subjects randomized to receive drug 1 matching placebo qd for 2 weeks (week 1-4 drug 1 matching placebo capsules; week 2-8 drug 1 matching placebo capsules)

Subjects randomized to open-label treatment:
5 subjects randomized to receive allopurinol 300 mg tablets qd for 2 weeks
   Cohort 2 approx 6 subjects randomized in 5:1 ratio treatment groups—drug+allopurinol:placebo+allopurinol

Subjects randomized to double-blind treatment:
5 subjects randomized to receive drug 1 200 mg qd (2 drug 1 100 mg capsules/day) for one week, followed by drug 1 400 mg qd (4 drug 1 100 mg capsules/day) for one week.
1 subject randomized to receive drug 1 matching placebo qd for 2 weeks (week 1-2 drug 1 matching placebo capsules; week 2-4 drug 1 matching placebo capsules).

All subjects receive open-label treatment with allopurinol 300 mg tablets qd for 4 weeks (one allopurinol tablet/day)

| | n | Drug regimen |
|---|---|---|
| Cohort 1 | | |
| Drug 1 | 10 | 200 mg qd for 1 week; 400 mg qd for 1 week |
| Placebo | 5 | Drug 1 placebo qd for 2 weeks |
| Active Control | 5 | 300 mg qd for 2 weeks |
| (allopurinol only) | | |
| Cohort 2 | | |
| Drug 1 + | 5 | 200 mg qd for 1 week; 400 mg qd for 1 week + |
| Allopurinol | | Allopurinol |
| Placebo + | 1 | Drug 1 placebo qd for 2 weeks + |
| Allopurinol | | Allopurinol |

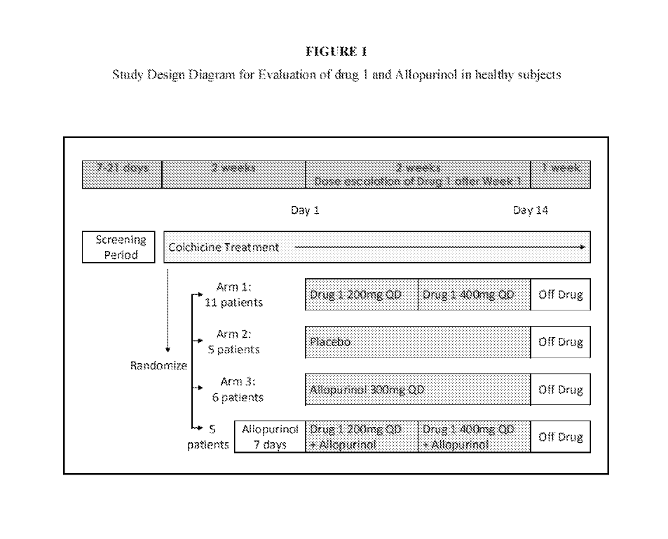

A summary of the study is shown in FIG. 1.

Dosing, Packaging & Dispensing

All subjects take colchicine 0.6 mg qd for 5 weeks.

For Cohorts 1 and 2, subjects randomized to double-blind treatment take drug 1 or drug 1 matching placebo every 24 hours (±1 h) (morning dose) for a period of 2 weeks.

For Cohort 1, subjects randomized to open-label treatment take allopurinol every 24 hours (±1 h) (morning dose) for a period of 2 weeks.

All subjects in Cohort 2 take allopurinol every 24 hours (±1 h) (morning dose) for a period of 4 weeks.

Subjects take the study medications or matching placebo with 240 mL water approximately 15-30 mins after a breakfast that does not contain fruit juice. Subjects are requested to then drink approximately 240 mL of water 1-2 hours after dosing.

Drug 1 or matching placebo is dispensed to randomized Drug 1/placebo groups on Days 1 and 8.

Drug 1 supplied as:
Cohort 1: size 2, 50 mg Immediate Release Capsules in 50-count bottles
Cohort 2: size 1, 100 mg Immediate Release Capsules in 20-count bottles
Matching placebo supplied as:
Cohort 1: size 2 gelatin capsules in 50-count bottles
Cohort 2: size 1 gelatin capsules in 20-count bottles

Allopurinol is dispensed to Cohort 1 subjects in the allopurinol group on Day 1.

Allopurinol is dispensed to Cohort 2 subjects on Day 7.

Allopurinol supplied as:

Cohort 1: 300 mg tablets in 100-count bottles
Cohort 2: 300 mg tablets in 100-count bottles

Subject Inclusion Criteria

Subjects must meet the following criteria to be eligible for the study:

1. Male or post-menopausal or surgically sterile female.
2. 18-75 years of age.
3. Hyperuricemic, defined as:
Cohort 1: Screening serum uric acid is ≧8 mg/dL
Cohort 2: Screening serum uric acid is ≧10 mg/dL
4. Subject meets one or more of the 1977 American Rheumatism Association (ARA) criteria for the diagnosis of acute arthritis of primary gout.
5. Subject is willing and able to give informed consent and adhere to visit/protocol schedules.

Screening

The following screening assessments are performed up to 4 weeks prior to the Baseline (Day 1) Visit:
Obtain signed, written informed consent
Demographics and baseline characteristics of gout
Record concomitant medications, medical/surgical history and concomitant diseases;
   confirm subject not infected with HIV;
   confirm subject has not taken any urate-lowering therapy (ULT) within the last 3 months or discontinued ULT due to toxicity or lack of efficacy
Review of inclusion and exclusion criteria
Physical examination, 12-lead ECG (triplicate—1-2 mins apart) and vital signs
Obtain blood samples for hematology, blood biochemistry (including sUA), hepatitis serology and serum pregnancy test (females)
Obtain urine samples for urinalysis

Randomization

A central randomization procedure is used to allocate subjects to treatment groups. Subjects are assigned a randomization number based on the time and date of the randomization request and randomization list. The randomization list is prepared using a validated program and occurs no later than 7 days prior to Baseline (Day 1). Following randomization, medication is shipped to the sites.

Blinding & Unblinding

The subjects, clinical staff and sponsor are blind to drug 1 or drug 1 matching placebo treatment. Subjects randomized to allopurinol in Cohort 1 and all subjects in Cohort 2 receive open-label medication.

The bioanalytical laboratory staff and the pharmacokineticist are authorized to break the code prior to the study ending to determine whether samples should be analyzed for drug 1. All other persons involved in the execution or evaluation of the trial remain blinded to the origin and results of the analyzed samples, until the database is locked, at the end of the study.

Schedule of Events

| | | Type of Visit | | | | | | | | | |
|---|---|---|---|---|---|---|---|---|---|---|---|
| | | Pre-Treatment | | | | | | | | | End of |
| | | Period | | | | | | | | | Study |
| | | Colchicine | Randomization | | | | | | | | End of Study |
| | | Dosing | (no visit), | Day 7*** | Treatment Period** | | | | | | (Day 21) |
| | Screening | (14 days prior | At least 7 days | Allopurinol | Day | Baseline | Day | Day | Day | Day | or Early |
| Time of Visit | Visit* | to Day 1) | prior to Day 1 | Dosing | 1 | (Day 1) | 8 | 9 | 14 | 15 | Termination |
| Informed consent | X | | | | | | | | | | |
| Demographic data | X | | | | | | | | | | |
| Medical & surgical history/ | X | | | | | | | | | | |
| Concomitant diseases | | | | | | | | | | | |
| Inclusion/exclusion criteria | X | | | | | X | | | | | |
| Physical examination | X | | | | | X | | | | X | X |
| Randomization procedure⁽¹⁾ | | | X | | | | | | | | |
| Dosing | | | | | | X | X | X | X | | |
| Colchicine treatment⁽²⁾ | | X | X | X | X | X | X | X | X | X | X |
| Allopurinol treatment | | | X | X | X | X | X | X | X | X | X |
| (Cohort 2 only) | | | | | | | | | | | |
| Urinalysis | X | | | | | X | X | | | X | X |
| Hematology | X | | | | | X | X | | | X | X |
| Blood biochemistry | X | | | | | X | X | | | X | X |
| including sUA | | | | | | | | | | | |
| Pregnancy Test⁽³⁾ | X | | | | | | | | | | |
| Hepatitis B & C Tests | X | | | | | | | | | | |
| HIV Test⁽¹⁰⁾ | X | | | | | | | | | | |
| ECG⁽¹²⁾ and vital signs | X | | | | | X | X | | | X | X |
| Concomitant medications | X | | | | X | X | X | X | X | X | X |
| Compliance | | | | | | | X | X | X | X | X⁽⁴⁾ |
| Record AEs | | | | | | X | X | X | X | X | X |
| Plasma sample for drug | | | | | | X⁽⁵⁾ | X⁽⁷⁾ | X⁽⁹⁾ | X⁽⁶⁾ | X⁽⁹⁾ | |
| exposure⁽¹¹⁾ | | | | | | | | | | | |
| 24 hr urine collections⁽⁸⁾ | | | | | X | X | X | X | X | X | |
| Additional sUA collections | | | | X | | X⁽⁵⁾ | X⁽⁵⁾ | X⁽⁹⁾ | X⁽⁶⁾ | | |

*Screening visit must be performed within 4 weeks prior to Baseline (Day 1).
**Visit dates are approximate (+/−2 Days).
***Allopurinol dosing begins only for subjects in Cohort 2.
⁽¹⁾Randomization procedure must be performed no later than 7 days prior to Baseline (Day 1).
⁽²⁾All subjects begin colchicine therapy 14 days prior to Baseline (Day 1) and discontinue colchicine therapy at the End of Study Visit (no dose taken at End of Study visit).
⁽³⁾Serum pregnancy test is conducted for female subjects at the Screening visit.
⁽⁴⁾Early Termination only.
⁽⁵⁾Samples collected 1 hour and 8 hours (+/−1 hour) post-dose in both Cohorts 1 and 2 as well as a pre-dose sample only for subjects in Cohort 2.
⁽⁶⁾Samples collected pre-dose and 8 hours (+/−1 hour) post-dose.
⁽⁷⁾Samples collected pre-dose, 1 hour and 8 hours (+/−1 hour) post-dose.
⁽⁸⁾24 hour urine collection should begin the mornings of: Day 1 and end pre-dose on Day 1, pre-dose on Day 8 and end pre-dose on Day 9, and pre-dose on Day 14 and end on Day 15. Samples should include all void within that time period for the determination of uric acid in urine and drug 1.
⁽⁹⁾Collected 24 hours post-dose.
⁽¹⁰⁾Only for subjects who cannot confirm their HIV status.
⁽¹¹⁾Subjects randomized to double-blind treatment only.
⁽¹²⁾Performed in triplicate approximately 1-2 minutes apart.

Day 7

Subjects in Cohort 2 given allopurinol 300 mg qd daily from Day 7 until the End of Study visit.

An additional sUA level is obtained on Day 7 for Cohort 2 subjects prior to the first dose of allopurinol.

Day 1
A 24-hour urine sample is collected (uric acid determinations and the excretion of drug 1) beginning the morning of Day 1
Continue administration of colchicine 0.6 mg qd
Continue administration of allopurinol started on Day 7 for subjects in Cohort 2 only

Baseline (Day 1) Visit

The following are completed for all subjects before dosing with study medication in the morning:
Collect 24-hour urine sample that was started on Day 1
Physical examination, ECG and vital signs
Record concomitant medications
Verification of the inclusion and exclusion criteria
Continue administration of colchicine 0.6 mg qd
Continue administration of allopurinol started on Day 7 for subjects in Cohort 2 only
Obtain blood samples for hematology and blood biochemistry (including sUA)
Collect urine sample for urinalysis
Plasma sample for drug exposure is collected pre-dose for Cohort 2 only
Breakfast not containing fruit juice
Study medication dosing with 240 mL of water approximately 15 to 30 minutes following breakfast

### After Dosing:

AEs are recorded, if applicable
Plasma samples collected 1 and 8 hours (+/−1) post-dose (double-blind subjects only) sUA sample collected 1 and 8 hours (+/−1) post-dose

Days 8, 9, 14 and 15

Each of the following are completed for all subjects before dosing on Days 8, 9, 14 and 15, in the morning:
Verification of compliance with study medication intake and interview for AEs
Continue administration of colchicine 0.6 mg qd
Continue administration of allopurinol started on Day 7 (Cohort 2 only)
Record concomitant medications
Breakfast not containing fruit juice
ECG and vital signs (Days 8, 15) and physical examination (Day 15)
Obtain blood samples for:
   Hematology and blood biochemistry (including sUA) (Days 8, 15)
   Drug 1 trough plasma concentration (Days 8, 14)
   sUA measurement (Days 9, 14)
   Plasma sample for drug exposure (Days 9, 15)
24-hour urine samples—initiate collection (Days 8, 14) and collect sample (Days 9, 15)
Collect urine sample for urinalysis (Days 8, 15)
Study medication administered with 240 mL water approx. 15-30 mins after breakfast (days 8, 9, 14)

After dosing, collect blood for:
Plasma sample 1 and 8 hours (+/−1) post-dose (double-blind subjects only) (Day 8)
Plasma sample 8 hours (+/−1) post-dose (double-blind subjects only) (Day 14)
sUA sample 1 and 8 hours (+/−1) post-dose (Day 8)
sUA sample 8 hours (+/−1) post-dose (Day 14)

End of Study Visit (Week 3; Day 21 or Early Termination)

The following are performed during the End of Study visit:
Verification of compliance with study medication intake and interview for AEs
Record concomitant medications
Physical examination, ECG and vital signs
Obtain blood samples for hematology and blood biochemistry (including sUA)
Collect urine sample for urinalysis
Discontinue colchicine and/or allopurinol treatment

Plasma Drug Samples

Plasma samples are analyzed using a validated LC-MS/MS analytical method. Blood samples are collected at each of the noted time points −4.5 mL for Cohort 1; 6 mL for Cohort 2.

Serum Uric Acid

Blood samples (4.5 mL) are collected and sUA levels determined at Screening (repeated as necessary prior to Day 1), Baseline (Day 1) (1 hour and 8 hours post-dose), Day 8 (trough, 1 hour and 8 hours post-dose), Day 9 (24 hours after Day 8 dose), and Day 14 (trough and 8 hours post-dose), Day 15 and End of Study. An additional sUA level is obtained on Day 7 for Cohort 2 subjects prior to the first dose of allopurinol.

Urinalysis & 24-Hour Urine Collections

Urinalysis (samples obtained at Screening, Baseline (Day 1), Day 8, Day 15, and End of Study) assessed by dipstick for pH, protein, glucose, specific gravity, and occult blood. If abnormal, microscopic examination for WBC, RBC and casts is performed. If trace protein is found, macroscopic examination for protein is performed. A qualitative analysis is done for ketones.

For 24-hour urine collection, subjects instructed to void their bladder prior to dosing; all urine output in the 24-hour period is collected. 24-hour urine samples used to measure uric acid and to measure excretion of drug 1 and of allopurinol/oxypurinol in Cohort 2, analyzed using a validated HPLC-tandem mass spectrometry (LC-MS/MS) analytical method.

Hematology & Biochemistry

Hematology assessments include: hemoglobin, hematocrit, red blood cell count (RBC), RBC parameters (mean corpuscular volume [MCV], mean corpuscular hemoglobin concentration [MCHC], mean corpuscular hemoglobin [MCH]), white blood cell count (WBC), white differential blood cell count (neutrophils, lymphocytes, monocytes, eosinophils, basophils) and platelet count. Hematology samples are obtained at Screening, Baseline (Day 1), Day 8, Day 15, and at the End of Study.

The biochemistry panel (fasting starting before or at midnight on the evening prior to the visit) include: total protein, glucose, albumin, alkaline phosphatase, ALT, AST, GGT, lactate dehydrogenase (LDH), direct and total bilirubin, amylase, lipase, calcium, phosphate, magnesium, sodium, potassium, BUN, chloride, sUA, creatinine, total cholesterol, low density lipoprotein, high density lipoprotein, aldosterone, Apo A-1, Apo B, and total triglycerides. Biochemistry samples are obtained at Screening, Baseline (Day 1), Day 8, Day 15, and at the End of Study.

Analysis Sets

Statistical analyses are performed using an intent-to-treat (ITT) analysis set, consisting of randomized subjects who took at least one dose of study medication.

Pharmacodynamics Assessment—Cohort 1

Primary pharmacodynamics parameter is the proportion of subjects with serum uric acid <6.0 mg/dL following 2 weeks of treatment. The ITT analysis set is used in the primary analysis of the primary pharmacodynamics parameter. The proportion of subjects with serum uric acid <6.0 mg/dL is compared between the treatment groups using Fisher's exact test. This global test of whether the proportions of subjects with sUA level <6.0 mg/dL are equal among treatment groups is followed by pairwise comparisons that compare each active group with the placebo group. No correction for multiple comparisons will be done. All treatment comparisons are to be considered exploratory. The proportions of subjects with sUA level <6.0 mg/dL in all other visits is regarded as a secondary endpoint. Secondary parameters include evaluating the:
proportion of subjects whose sUA levels are <6.0 mg/dL, <5.0 mg/dL and <4.0 mg/dL at each visit;
absolute and percent reduction from baseline in sUA levels at each visit;
maximum percent reduction in sUA levels from baseline during the entire treatment period;
percent change in 24-hour urine uric acid level from baseline to Day 15;
safety and tolerability of drug 1 in subjects with gout;
pharmacokinetics, safety and tolerability of drug 1 in combination with allopurinol in subjects with gout.

Pharmacodynamics Assessment—Cohort 2

Primary pharmacodynamic endpoint is the percent reduction from baseline in sUA levels following 2 weeks of continuous treatment with drug 1 in combination with allopurinol. Pharmacokinetics, safety and tolerability in combination with allopurinol are also assessed. The treatment effects on continuous parameters measured in multiple visits investigated by means of analysis of covariance (ANCOVA) with baseline value and treatment group as covariates. For binary data, Fisher's exact test is used to test the null hypothesis that the proportions of endpoints among treatment groups are equal. As in the analysis of the primary endpoint, each dose group will be compared to the placebo group.

Safety Parameters: Vital Signs, Physical Examination & Electrocardiogram (ECG)

Vital signs (temperature, systolic and diastolic blood pressure (mmHg), pulse rate, and respiratory rate) are collected at Screening, Baseline (Day 1), Day 8, Day 15, and End of Study visits.

A complete physical examination, including weight, is performed at Screening, Baseline (Day 1), Day 15, and at the End of Study visit. Body weight and body mass index (BMI) are analyzed descriptively; as actual values and as changes from baseline. Any physical examination abnormalities are recorded.

Safety ECGs (12-lead ECGs recorded at 25 mm/s and reporting ventricular rate and PR, RR, QRS, QT and QTc intervals) are recorded in triplicate approximately 1-2 minutes apart with no more than 5 minutes total for all ECGs. ECGs interpreted immediately at all visits. Changes from baseline QT and QTc interval are monitored on an ongoing basis throughout the study. ECGs performed after subject has been in the supine position for at least 10 minutes at the Screening, Baseline (Day 1), Day 8, Day 15, and End of Study visits.

Adverse Events

An AE is any untoward medical occurrence in a subject administered a pharmaceutical product and which does not necessarily have a causal relationship with this treatment. An AE can therefore be any unfavorable and unintended sign (including an abnormal laboratory finding), symptom, or disease temporally associated with the use of a medicinal (investigational) product, whether or not related to the medicinal (investigational) product. All AEs are recorded. Abnormal laboratory values do not themselves represent AEs unless they are indicative of a disease or defect and/or necessitate intervention. AEs with new onset after the initiation of study medication or AEs that increase in intensity or severity during the Treatment Periods of the study are considered treatment-emergent AEs. Adverse events are monitored during the study and analyzed with respect to overall incidence, severity and potential relationship of the AEs to the study medication. Adverse events with onset after the first administration of the study medication are considered treatment-emergent, including any AE with onset prior to initiation of study medication and increased severity after the treatment initiation.

Adverse events associated with gout are tabulated separately, split over 3 analysis phases: screening (before the first medication intake), treatment (from first until last medication intake, plus 1 day) and follow-up (the remainder of the study period, until trial termination). The Investigator should assess the severity of the AE and the relationship of the AE to the study medication:
MILD Subject is aware of sign or symptom, but it is easily tolerated
MODERATE Discomfort enough to cause interference with the subject's usual activities
SEVERE Incapacitating with inability of the subject to work or perform usual activities
NOT RELATED AE is not related to the use of the medication.
UNLIKELY AE for which an alternative explanation is more likely, e.g., concomitant medication(s) or concomitant disease(s)
POSSIBLE AE might be due to the use of the medication. An alternative explanation, e.g., concomitant medication(s) or concomitant disease(s), is inconclusive.

Serious Adverse Events (SAE)

A SAE results in any of the following:

Death.

Life-threatening adverse experience.

Hospitalization (unplanned hospital stay) or prolongation of existing hospitalization.

Persistent or significant disability/incapacity.

Congenital anomaly/birth defect.

Subjects experiencing a SAE or an emergency situation will be examined by a physician as soon as possible. The physician in attendance will do whatever is medically needed for the safety and well-being of the subject. A written report for a SAE must follow within 24 hours of knowledge.

### Example 2

Drug 1 was tested according to the clinical trial protocol described in example 1. Actual enrollment was as follows:

Cohort 1: 21 subjects −11 randomized to Drug 1
−5 randomized to placebo
−5 randomized to open-label allopurinol
   Cohort 2: 6 subjects −5 randomized to Drug 1+allopurinol
−1 to placebo+allopurinol

### Preliminary Safety Summary (Cohort 1 Only)

Drug 1 was well tolerated in this study, with no SAEs, deaths or discontinuations due to adverse events and no clinically significant changes in physical exam findings or vital signs. No clinically significant ECG findings including interval measurements, and no dose-related increase in adverse events (all events were transient and mild to moderate in severity).

Two patients had >30% increase in serum creatinine (SCr) while on 400 mg QD (Grade 1 AE) with no associated increase in BUN levels and no significant abnormality in urinalysis; SCr quickly fell back to normal limits after patients finished the study and no consistent changes in other laboratory parameters in these patients was observed. One subject on allopurinol experienced hyperaldosteronemia and two subjects experienced abdominal pain.

| Treatment | Responders¹ Day 14 |
|---|---|
| Drug 1 | 6/10² |
| Placebo | 0/5 |
| Allopurinol | 5/5 |

¹Defined as those with sUA < 6 mg/dL
²One over-producer excluded from analysis

Drug 1 plasma levels in gout patients were generally consistent with those observed in Phase 1 healthy volunteer studies. On average, Drug 1-treated patients achieved a 40% reduction in serum urate levels after the first week of treatment. Two patients randomized to Drug 1 had a baseline sUA above 11 mg/dL; none of the allopurinol patients had baseline values this high. The two other patients not below 6 mg/dL at Day 14 were at 6.2 and 6.3 mg/dL.

### Example 3

Drug 1 is evaluated according to the clinical trial protocol described in example 1, using BCX4208 (is 7-(((3R,4R)-3-hydroxy-4-(hydroxymethyl)pyrrolidin-1-yl)methyl)-3H-pyrrolo[3,2-d]pyrimidin-4(5H)-one) in place of allopurinol.

### Example 4

Drug 1 is evaluated according to the clinical trial protocol described in example 1, using probenecid in place of allopurinol.

### Example 5

Drug 1 is evaluated according to the clinical trial protocol described in example 1, using tranilast in place of allopurinol.

### Example 6

Drug 1 is evaluated according to the clinical trial protocol described in example 1, using sulfinpyrazone in place of allopurinol.

### Example 7

Drug 1 is evaluated according to the clinical trial protocol described in example 1, using losartan in place of allopurinol.

### Example 8

Drug 1 is evaluated according to the clinical trial protocol described in example 1, using fenofibrate in place of allopurinol.

### Example 9

Drug 1 is evaluated according to the clinical trial protocol described in example 1, using benzbromarone in place of allopurinol.

### Example 10

Study Objectives
To compare the multiple-dose pharmacokinetics of febuxostat in the absence versus presence of drug 1 co-administration.
To compare the multiple-dose pharmacokinetics of drug 1 in the absence versus presence of febuxostat co-administration.
To measure the effects of drug 1 and febuxostat, both alone and in combination, on serum uric acid concentrations and amounts of urate excreted into urine.
To evaluate the safety and tolerability of multiple-doses of drug 1 and febuxostat, both alone or in combination.

Study Details

The study is a two or three-panel, placebo-controlled (for drug 1), double-blinded (for drug 1 and matched placebo), randomized, cross-over study with up to 54 healthy adult subjects. Each panel consisting of 18 subjects is sequentially enrolled, starting with Panel 1 (200 mg once daily dose of drug 1 or placebo), followed by Panel 2 (400 mg once daily dose of drug 1 or placebo), and followed by optional Panel 3 (100 mg to 600 mg once daily dose of drug 1 or placebo).

The placebo control is included in this study to better assess safety and tolerability and the serum urate lowering effect of drug 1 in combination with febuxostat. Serum urate levels may be influenced by frequent blood draws and meal contents; therefore, the true effect on serum urate levels can be determined by correcting for placebo (subtract serum urate effect in placebo subjects from serum urate effect in active subjects).

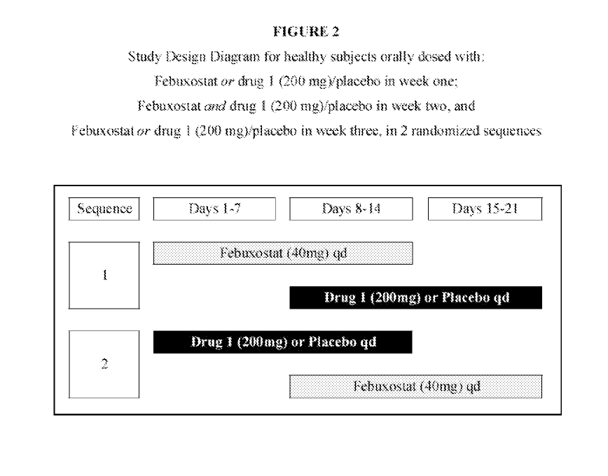

Subjects in each panel are randomly assigned to one of two treatment sequences (Sequence A or B) after all Baseline (Day −1) procedures have been completed. Each treatment sequence within a panel consists of 9 subjects randomly assigned to receive drug 1 (6 of 9 subjects) or matching placebo (3 of 9 subjects) in a double-blinded fashion. For each treatment sequence, single-agent drug treatment is administered on Days 1 to 7 (either open-label febuxostat or double-blinded drug 1 or matched placebo by random assignment), followed by combination treatment on Days 8 to 14, and completing with the alternative single-agent drug treatment on Days 15 to 21, as follows (Panel 1 shown in FIG. 2):

### Panel 1 (200 mg Drug 1)

Sequence A: Days 1-7: Febuxostat, 40 mg (once daily)
Days 8 to 14: drug 1, 200 mg or placebo in combination with febuxostat (once daily)
Days 15-21: drug 1, 200 mg, or placebo (once daily)
   Sequence B: Days 1-7: drug 1, 200 mg, or placebo (once daily)
Days 8 to 14: drug 1, 200 mg or placebo in combination with febuxostat (once daily)
Days 15-21: Febuxostat, 40 mg (once daily)

### Panel 2 (400 mg Drug 1)

Sequence A: Days 1-7: Febuxostat, 40 mg (once daily)
Days 8 to 14: drug 1, 400 mg or placebo in combination with febuxostat (once daily)
Days 15-21: drug 1, 400 mg, or placebo (once daily)
   Sequence B: Days 1-7: drug 1, 400 mg, or placebo (once daily)
Days 8 to 14: drug 1, 400 mg or placebo in combination with febuxostat (once daily)
Days 15-21: Febuxostat, 40 mg (once daily)

### Panel 3, Optional (Selected Dose of Drug 1)

Sequence A: Days 1-7: Febuxostat, 40 mg (once daily)
Days 8 to 14: drug 1, 100-600 mg or placebo in combination with febuxostat (once daily)
Days 15-21: drug 1, 100-600 mg, or placebo (once daily)
   Sequence B: Days 1-7: drug 1, 100-600 mg, or placebo (once daily)
Days 8 to 14: drug 1, 100-600 mg or placebo in combination with febuxostat (once daily)
Days 15-21: Febuxostat, 40 mg (once daily)
   Study drug(s) are administered to each subject every morning, at approximately the same time of day, 0-15 minutes after finishing a full breakfast (at least 620 Kcal); breakfast standardized on Days −1, 7, 14, and 21.

Blood Samples

Serial blood samples for PK and PD assessments are collected up to 24 hours after dosing on Days 7, 14, and 21. Urine (total catch) for assays of drug 1, creatinine, and uric acid are collected starting on Day −1 (pre-treatment baseline) and after dosing on Days 7, 14, and 21. Final safety assessments are completed when subjects return to the clinic at 7±1 days after discharge from the clinic on Day 22 (Follow-Up visit on Day 29). The total volume of blood collected from each subject is approximately 518 mL.

Duration

Subjects receive a once daily treatment with one or two study drugs (febuxostat and/or drug 1/placebo) for 21 days. The total duration of study including screening period is approximately 4 to 9 weeks for an individual subject.

Patient Population & Inclusion Criteria

Screening procedures to determine subject eligibility are performed 28 days before the first dose of study drug (Day 1). A total of up to 54 subjects enrolled in up to 3 panels consisting of 2 sequences each, with 9 subjects per sequence (18 subjects per panel). Subjects withdrawing after dosing are not replaced. Subjects must meet the following criteria to be eligible for the study:
Healthy adults ≧18 and ≦65 years of age.
Male, post-menopausal or surgically sterile female.
Screening serum urate level ≧5-6 mg/dL (357 mmol/L).
Body weight >50 kg (110 lbs) and body mass index (BMI) 18 and 30 kg/m².
Free of any clinically significant disease and laboratory parameters (chemistry, hematology, and urinalysis) within normal limits (except for serum urate).
Normal or clinically acceptable physical examination and no clinically relevant abnormalities in blood pressure, heart rate, body temperature or respiratory rate.

Drug Formulation

Drug 1 Immediate Release capsules, 100 mg, matching placebo. Drug 1, placebo, and ULORIC® formulations require no special handling.

Dosage Regimen

Subjects in Panel 1 receive a 200 mg dose of drug 1 or placebo once daily for 14 days and a 40 mg dose of febuxostat once daily for 14 days. Subjects in Panel 2 receive a 400 mg dose of drug 1 or placebo once daily for 14 days and a 40 mg dose of febuxostat once daily for 14 days. Subjects in Panel 3 receive a 100-600 mg dose of drug 1 or placebo once daily for 14 days and a 40 mg dose of febuxostat once daily for 14 days. Subjects in Sequence A begin dosing on Day 1 with febuxostat and take their first dose of drug 1 or placebo on Day 8. Subjects in Sequence B begin dosing on Day 1 with drug 1 or placebo and take their first dose of febuxostat on Day 8.

### Example 11

Drug 1 was tested according to the clinical trial protocol described in Example 10.

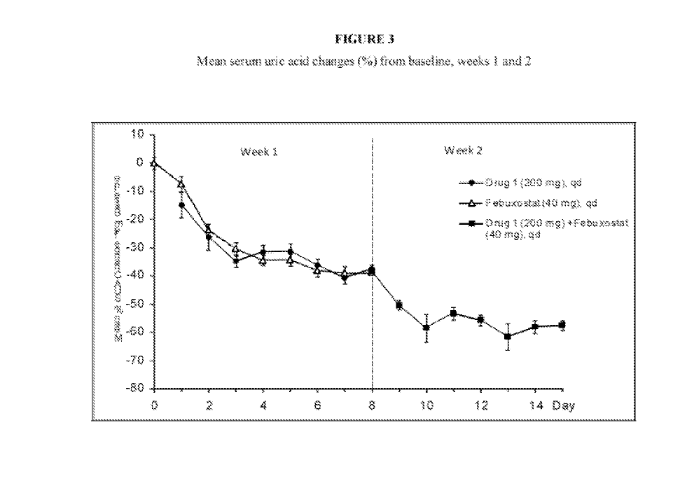

14 volunteers were enrolled for panel 1 (200 mg drug 1), randomized into sequence 1, administered drug (n=5) or placebo (n=2); or sequence 2, administered Febuxostat (n=7). Serum uric acid levels were determined as described in Example 1 and Example 10 and the % serum uric acid changes, (using Day 1, −24 h as 100%), days 1-15 are shown in FIG. 3 and in the table below (excluding placebo values).

| | | | | | | Drug 1 + | |
|---|---|---|---|---|---|---|---|
| | | Febuxostat | | Drug 1 | | Febuxostat | |
| | | Mean | SE | Mean | SE | Mean | SE |
| Week 1 | Day 1 | −7.5 | 2.19 | −14.8 | 4.60 | | |
| | Day 2 | −23.8 | 2.79 | −26.3 | 4.60 | | |
| | Day 3 | −30.5 | 2.27 | −34.7 | 2.26 | | |
| | Day 4 | −34.5 | 2.38 | −31.6 | 2.54 | | |
| | Day 5 | −34.3 | 1.86 | −31.3 | 2.82 | | |
| | Day 6 | −38.1 | 2.19 | −36.2 | 2.20 | | |
| | Day 7 | −39.0 | 2.17 | −40.7 | 1.97 | | |
| Week 2 | Day 8 | −38.9 | 2.34 | −37.6 | 1.54 | −38.2 | 1.3 |
| | Day 9 | | | | | −50.4 | 1.7 |
| | Day 10 | | | | | −58.5 | 4.9 |
| | Day 11 | | | | | −53.4 | 2.2 |
| | Day 12 | | | | | −55.7 | 1.9 |
| | Day 13 | | | | | −61.6 | 4.6 |
| | Day 14 | | | | | −58.0 | 2.2 |
| | Day 15 | | | | | −57.5 | 1.7 |

### Example 12

Drug 1 was evaluated according to the clinical trial protocol described in Example 10.

In each panel, 18 subjects were randomly assigned to receive either Drug 1 or matching placebo randomized in a 2:1 ratio or 40 mg of febuxostat qd during the first week as single agents, the combination of the two agents in the second week and finally, the alternative single agent in the third week. Serum urate levels were evaluated daily.

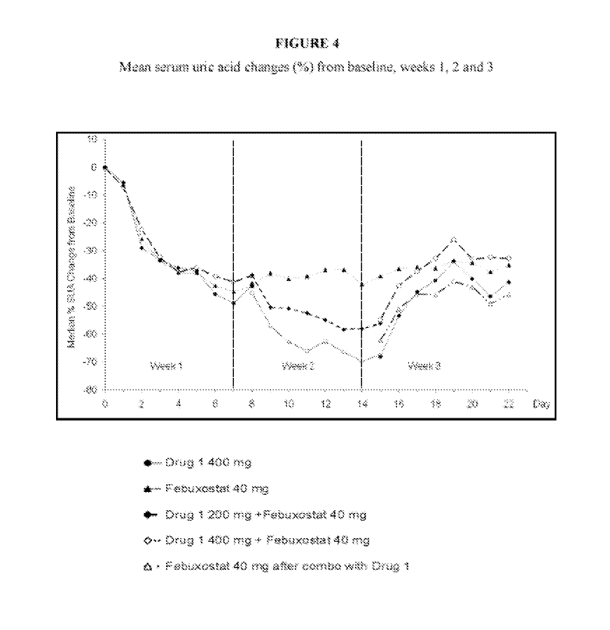

Serum uric acid levels were determined as described in Example 1 and Example 10 and the serum uric acid changes (absolute and %) from baseline, (using Day 1, −24 h as 100%), weeks 1, 2 and 3 are presented in the tables below (excluding placebo values) and graphically in FIG. 4.

Absolute sUA reduction from baseline, mg/dL (mg/dL±SE, N)

| | Week 1 | | Week 2 | | Week 3 | |
|---|---|---|---|---|---|---|
| | | Intraday | | Intraday | | Intraday |
| Treatment | Trough | peak | Trough | peak | Trough | peak |
| Drug 1 (200 mg) wk1 | −2.4 ± | −3.1 ± | −3.2 ± | −4.2 ± | −2.0 ± | −2.8 ± |
| & wk3, combo on | 0.13 (6) | 0.16 (6) | 0.21 (12) | 0.21 (12) | 0.2 (6) | 0.21 (6) |
| wk2 | | | | | | |
| Drug 1 (400 mg) wk1 | −3.4 ± | −4.2 ± | −4.5 ± | −5.4 ± | −2.9 ± | −3.6 ± |
| & wk3, combo on | 0.36 (6) | 0.38 (6) | 0.43 (12) | 0.43 (12) | 0.25 (6) | 0.24 (6) |
| wk2 | | | | | | |
| Febuxostat (40 mg) | −2.8 ± | −3.4 ± | −2.6 ± | −3.1 ± | −2.7 ± | −3.3 ± |
| alone wk1 through | 0.14 (18) | 0.13 (18) | 0.25 (6) | 0.22 (6) | 0.19 (17) | 0.17 (17) |
| wk3 | | | | | | |

Percent sUA reduction from baseline, % (%±SE, N)

| | Week 1 | | Week 2 | | Week 3 | |
|---|---|---|---|---|---|---|
| | | Intraday | | Intraday | | Intraday |
| Treatment | Trough | peak | Trough | peak | Trough | peak |
| Drug 1 | −39.7 ± | −50.4 ± | −58.4 ± | −73.9 ± | −34.7 ± | −48.5 ± |
| (200 mg) | 1.9 (6) | 2.9 (6) | 2.0 (12) | 1.1 (12) | 2.1 (6) | 1.6 (6) |
| wk1 & wk3, | | | | | | |
| combo on wk2 | | | | | | |
| Drug 1 | −48.5 ± | −60.7 ± | −68.9 ± | −81.5 ± | −45.5 ± | −56.9 ± |
| (400 mg) | 1.9 (6) | 1.4 (6) | 2.0 (12) | 1.1 (12) | 3.0 (6) | 2.7 (6) |
| wk1 & wk3, | | | | | | |
| combo on wk2 | | | | | | |
| Febuxostat | −45.4 ± | −55.3 ± | −41.2 ± | −50.1 ± | −42.1 ± | −49.0 ± |
| (40 mg) alone | 1.8 | 1.5 (18) | 4.2 (6) | 3.8 (6) | 2.0 | 3.3 |
| wk1 through | (18) | | | | (17) | |
| wk3 | | | | | | |

| | | | | | Drug 1 | Drug 1 | FBX |
|---|---|---|---|---|---|---|---|
| | | | | | 200 mg + | 400 mg + | 40 mg + |
| | | Drug 1 | Drug 1 | FBX | FBX | FBX | after |
| | | 200 mg | 400 mg | 40 mg | 40 mg | 40 mg | combo |
| Week 1 | Day 1 | −7.4 | −5.5 | −6.4 | | | |
| | Day 2 | −22.3 | −28.9 | −25.7 | | | |
| | Day 3 | −32.4 | −33.3 | −33.6 | | | |
| | Day 4 | −37.7 | −36.2 | −37.8 | | | |
| | Day 5 | −36 | −37.3 | −37.9 | | | |
| | Day 6 | −39.1 | −45.6 | −42.6 | | | |
| | Day 7 | −41.2 | −48.8 | −44.6 | | | |
| Week 2 | Day 8 | −38.9 | −42.5 | −42.7 | −38.9 | −45.2 | |
| | Day 9 | | | −38.1 | −50.4 | −57 | |
| | Day 10 | | | −40 | −50.8 | −62.8 | |
| | Day 11 | | | −39.2 | −52.4 | −66 | |
| | Day 12 | | | −36.9 | −54.9 | −62.6 | |
| | Day 13 | | | −36.8 | −58.3 | −66.4 | |
| | Day 14 | | | −42 | −58 | −69.8 | |
| Week 3 | Day 15 | −54.8 | −68.1 | −39.1 | −56.2 | −67.4 | −62.3 |
| | Day 16 | −42.5 | −53.4 | −36.5 | | | −50.9 |
| | Day 17 | −37.5 | −44.8 | −35.8 | | | −45.6 |
| | Day 18 | −32.8 | −40.7 | −36.3 | | | −45.7 |
| | Day 19 | −25.9 | −33.8 | −33.6 | | | −41 |
| | Day 20 | −33 | −40 | −34.3 | | | −42.9 |
| | Day 21 | −32.4 | −46.4 | −37.6 | | | −49.1 |
| | Day 22 | −32.8 | −41.3 | −35.2 | | | −45.7 |

### Example 13

Drug 1 is evaluated according to the clinical trial protocol described in Example 10, using BCX4208 (is 7-(((3R,4R)-3-hydroxy-4-(hydroxymethyl)pyrrolidin-1-yl)methyl)-3H-pyrrolo[3,2-d]pyrimidin-4(5H)-one) in place of febuxostat.

### Example 14

Drug 1 is evaluated according to the clinical trial protocol described in Example 10, using probenecid in place of febuxostat.

### Example 15

Drug 1 is evaluated according to the clinical trial protocol described in Example 10, using tranilast in place of febuxostat.

### Example 16

Drug 1 is evaluated according to the clinical trial protocol described in Example 10, using sulfinpyrazone in place of febuxostat.

### Example 17

Drug 1 is evaluated according to the clinical trial protocol described in Example 10, using losartan in place of febuxostat.

### Example 18

Drug 1 is evaluated according to the clinical trial protocol described in Example 10, using fenofibrate in place of febuxostat.

### Example 19

Drug 1 is evaluated according to the clinical trial protocol described in Example 10, using benzbromarone in place of febuxostat.

### Example 20

Drug 1 was tested according to the clinical trial protocol described in Example 1, with subject enrollment was as follows:

Cohort 1: 21 subjects −11 randomized to Drug 1 (one over-producer was excluded from analysis);
−5 randomized to placebo
−5 randomized to open-label allopurinol

Of the 10 subjects randomized to Drug 1:

two were considered to have moderate diminished renal function, defined as creatinine clearance (CrCL) from >30 mL/min to <60 mL/min; and

three were considered to have mild diminished renal function, defined as creatinine clearance (CrCL) from 60 mL/min to 80 mL/min.

Note that in general, about 30% of the gout patient population demonstrate mild to moderate diminished renal function (“renal impairment”).

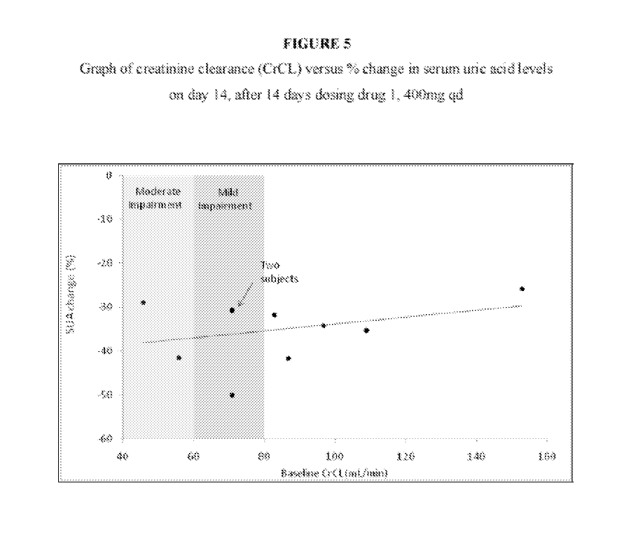

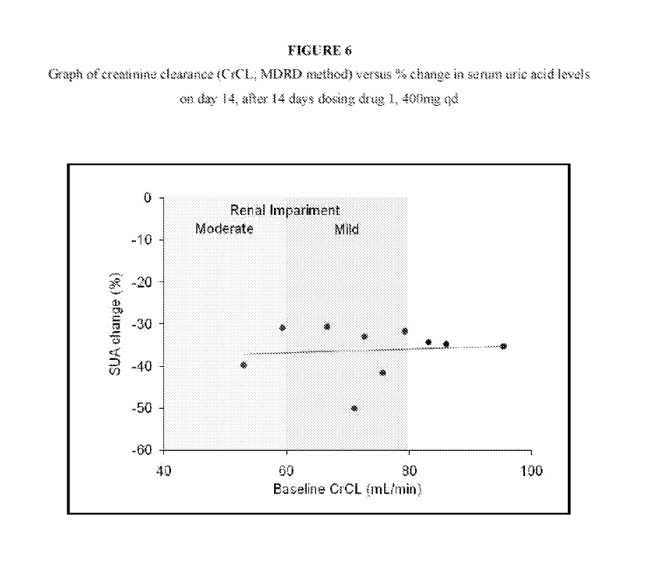

Baseline CrCl was calculated by both Cockcroft-Gault and MDRD methods FIG. 5 presents a graph of creatinine clearance (CrCL) versus % change in serum uric acid levels on day 14, after 14 days dosing drug 1, 400 mg qd. The MDRD method identified three additional patients with some level of renal impairment as shown in FIG. 6. With both methods, the slope of the linear trend line for sUA reduction with Drug 1 shows an increasing benefit with lower CrCL.

All patients with renal impairment experienced at least a 30% reduction in sUA by the end of week 2; the patient with the lowest baseline CrCl (53 ml/min) had a 40% reduction in sUA.

### Example 21

Study Objectives
To measure the effect of drug 1, alone and in combination with allopurinol, on serum urate concentration.
To evaluate the safety and tolerability of drug 1 in combination with allopurinol.

Methodology

The study is open-label in gout patients with hyperuricemia, involving 2 panels of −10-12 patients per panel. The panels may be enrolled simultaneously.

Panel 1 receives a once-daily dose of allopurinol 300 mg alone for 7 days and for an additional 7 days in combination with a once-daily dose of drug 1 400 mg. Patients then receive 7 days of a once-daily dose of drug 1 400 mg alone.

Panel 2 follows the same regimen as panel 1, at doses of allopurinol 300 mg and drug 1 600 mg.

### Panel 1

Day −10 to Day 28: colchicine prophylaxis; or
Day −7 to Day 28: colchicine prophylaxis (for patients not washing out from ULT)
Days 1-7: allopurinol 300 mg qd
Days 8-14: drug 1 400 mg qd+allopurinol 300 mg qd
Days 15-21: drug 1 400 mg qd

### Panel 2

Day −10 to Day 28: colchicine prophylaxis; or
Day −7 to Day 28: colchicine prophylaxis (for patients not washing out from ULT)
Days 1-7: allopurinol 300 mg qd
Days 8-14: drug 1 600 mg qd+allopurinol 300 mg qd
Days 15-21: drug 1 600 mg qd

| | Days 1-7 | Days 8-14 | Days 15-21 |
|---|---|---|---|
| PANEL 1 | | | |
| allopurinol | 300 mg | 300 mg | |
| drug 1 | | 400 mg | 400 mg |
| PANEL 2 | | | |
| allopurinol | 300 mg | 300 mg | |
| drug 1 | | 600 mg | 600 mg |

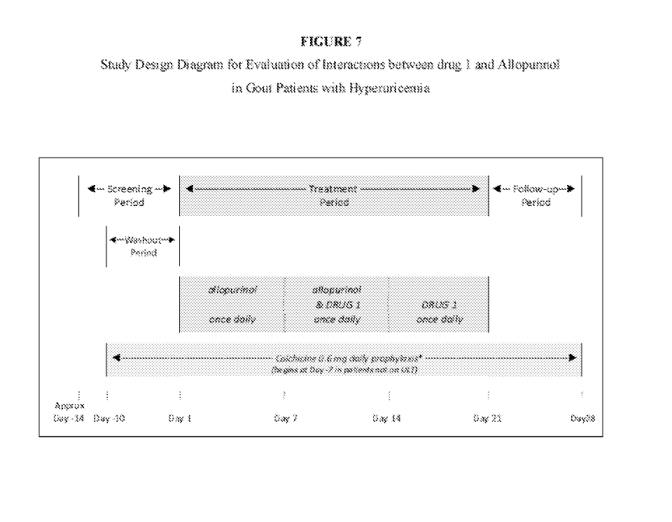

A detailed study design diagram is shown in FIG. 7.

Gout patients on a urate lowering therapy (ULT), have previously taken a ULT or who have never taken a ULT are eligible to participate. Screening procedures to determine patient eligibility are performed within approximately 14 days of the first dose of study drug (Day 1). Subjects currently on ULT wash-out for at least 10 days before Day 1, and begin colchicine (Colcrys® 0.6 mg qd, URL Pharma) as prophylaxis for gout flares at the beginning of the wash-out period. Subjects not on ULT begin colchicine administration at least 7 days prior to Day 1. Patients intolerant of colchicine during the screening period are considered screen failures and not enrolled. Prophylactic administration of colchicine continues for 7 days after the last dose of study medication.

Eligible patients report to the study center early morning on Days −1, 7, 14, and 21 (or the afternoon/evening the day before). Serial blood samples are collected on Days −1, 7, 14, and 21.

Study medication is administered to each patient and taken orally every morning with approximately 240 mL of water, at approximately the same time of the morning every day, and approximately 30 minutes after finishing a full breakfast (approximately 650 Kcal and 35% fat); the breakfast is standardized on Days −1 to 21.

Number of Patients

Up to 24 patients enrolled in 2 panels (up to 12 patients per panel).

Dosage and Administration

Drug 1 Capsules 100 mg and allopurinol tablets 300 mg are provided for the study. Colchicine (Colcrys®, URL Pharma) is provided by the study site. Drug 1, allopurinol and Colchicine are taken orally.

Duration of Treatment

The duration of participation per patient is approximately 49 days, including up to a 14 day Screening Period, 21 days of study treatment, 7 days of post-treatment colchicine prophylaxis and concluding with a final Follow-up Visit (Day 28).

Inclusion Criteria
Patient is male or post-menopausal or surgically sterile female.
Patient is 18-80 years of age.
Patient meets one or more criteria for the diagnosis of gout as per the American Rheumatism Association (ARA) Criteria for the Classification of Acute Arthritis of Primary Gout (Appendix B).
Patient sUA ≧8 mg/dL at screening.
Patient willing and able to give informed consent and adhere to visit/protocol schedules
No clinically relevant abnormalities in blood pressure (BP), heart rate (HR), body temperature and respiratory rate as per Investigator's judgment.

Evaluation Criteria & Pharmacodynamic Evaluations

Subject urate serum levels are recorded, and any safety concerns assessed (by adverse events, clinical laboratory test results, vital signs, 12-lead ECGs, and physical examinations). The analysis of pharmacodynamic data includes all study participants receiving study drug and demonstrating evaluable data. Urate serum concentrations are evaluated for treatment- and/or time-dependent changes relative to baseline (Day −1), and may be expressed both in standard units (mg/dL) and as changes from baseline (e.g., percent change, absolute change, maximal change, and time or day of maximal change).

### Example 22

Drug 1 was tested according to the protocol described in Example 21.

Drug 1 in combination with allopurinol was well tolerated, with no SAEs, deaths or discontinuations due to adverse events.

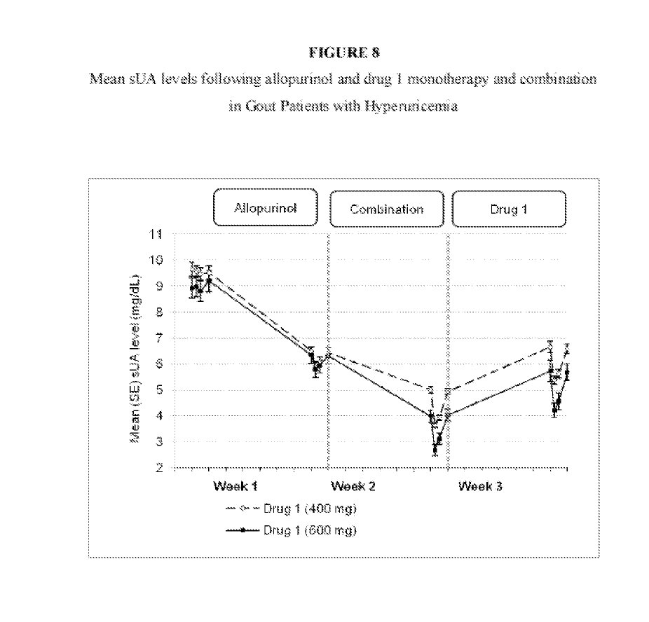

Serum urate levels were measured and absolute and % reduction levels from baseline calculated, for Allopurinol monotherapy, Drug 1 monotherapy and Allopurinol plus Drug 1 combination. Mean levels, mean absolute reductions (mg/dL, all data SE) and % change results are presented in the table below and in FIG. 8.

| Drug 1 | | | Allopurinol | | Drug 1 |
|---|---|---|---|---|---|
| (mg) | | Baseline | 300 mg | Combination | alone |
| 400 | Mean sUA level | 9.8 ± 0.27 | 6.5 ± 0.16 | 5.0 ± 0.11 | 6.6 ± 0.22 |
| | Mean absolute | 0 | −3.1 ± 0.28 | −4.6 ± 0.34 | −3.0 ± 0.46 |
| | sUA reduction | | | | |
| | Mean sUA % | 0 | −32.2 ± 2.1 | −47.6 ± 2.2 | −30.2 ± 3.7 |
| | change | | | | |
| 600 | Mean sUA level | 9.1 ± 0.38 | 6.3 ± 0.31 | 4.0 ± 0.24 | 5.7 ± 0.41 |
| | Mean absolute | 0 | −2.6 ± 0.26 | −4.9 ± 0.23 | −3.3 ± 0.23 |
| | sUA reduction | | | | |
| | Mean sUA % | 0 | −28.7 ± 2.4 | −55.5 ± 1.6 | −37.2 ± 2.6 |
| | change | | | | |

% Response rate for sUA falling below 6, 5 or 4 mg/dL following Allopurinol Monotherapy, Drug 1 Monotherapy and Allopurinol plus drug 1 combination are presented in the table below.

| Drug 1 | | Allopurinol | | Drug 1 |
|---|---|---|---|---|
| (mg) | Baseline | 300 mg | Combination | alone |
| 400 | | | | |
| (<6 mg/dL) | 0% | 10% | 100% | 20% |
| (<5 mg/dL) | 0% | 0% | 50% | 0% |
| (<4 mg/dL) | 0% | 0% | 0% | 0% |
| 600 | | | | |
| (<6 mg/dL) | 0% | 30% | 100% | 67% |
| (<5 mg/dL) | 0% | 10% | 90% | 33% |
| (<4 mg/dL) | 0% | 0% | 50% | 0% |

### Example 23

Study Objectives
To measure the effect of febuxostat alone and in combination with drug 1 on serum urate concentrations.
To evaluate the safety and tolerability of drug 1 in combination with febuxostat.

Methodology

The study is open-label in gout patients with hyperuricemia, involving 2 panels of −10-12 patients per panel. The panels may be enrolled simultaneously.

Panel 1 receives a once-daily dose of febuxostat 40 mg with ascending doses of drug 1.

Panel 2 receives a once-daily dose of febuxostat 80 mg with ascending doses of drug 1.

### Panel 1 (Febuxostat 40 mg)

Day −14 to Day 28: colchicine prophylaxis; or
Day −7 to Day 28: colchicine prophylaxis (for patients not washing out from ULT)
Days 1-7: febuxostat 40 mg qd
Days 8-14: febuxostat 40 mg qd+drug 1 400 mg qd
Days 15-21: febuxostat 40 mg qd+drug 1 600 mg qd

### Panel 2 (Febuxostat 80 mg)

Day −14 to Day 28: colchicine prophylaxis; or
Day −7 to Day 28: colchicine prophylaxis (for patients not washing out from ULT)
Days 1-7: febuxostat 80 mg qd
Days 8-14: febuxostat 80 mg qd+drug 1 400 mg qd
Days 15-21: febuxostat 80 mg qd+drug 1 600 mg qd

| | Days 1-7 | Days 8-14 | Days 15-21 |
|---|---|---|---|
| PANEL 1 | | | |
| febuxostat | 40 mg | 40 mg | 40 mg |
| drug 1 | | 400 mg | 600 mg |
| PANEL 2 | | | |
| febuxostat | 80 mg | 80 mg | 80 mg |
| drug 1 | | 400 mg | 600 mg |

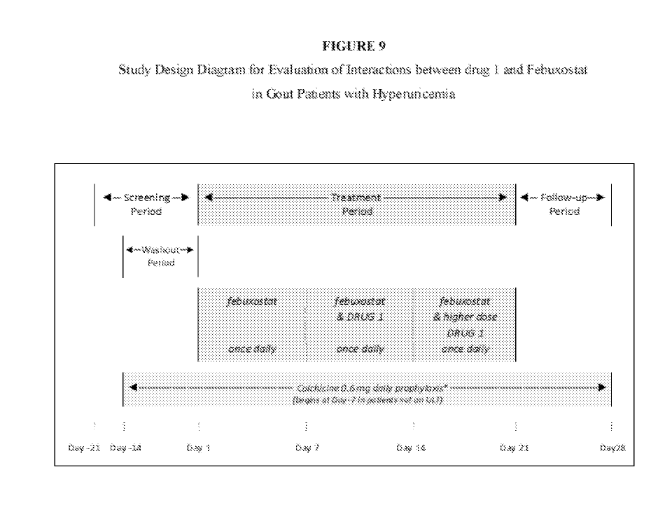

A detailed study design diagram is shown in FIG. 9.

Gout patients on a urate lowering therapy (ULT), have previously taken a ULT or who have never taken a ULT are eligible to participate. Screening procedures to determine patient eligibility are performed within approximately 21 days of the first dose of study drug (Day 1). Subjects currently on ULT wash-out for approximately 14 days before Day 1, and begin colchicine (Colcrys® 0.6 mg qd, URL Pharma) as prophylaxis for gout flares. During the washout period, subjects who have discontinued ULT may have their sUA re-tested (at least 7 days after washing out) to confirm eligibility. Subjects not on ULT begin colchicine administration at least 7 days prior to Day 1. Patients demonstrating an increase in CPK >5×ULN discontinue colchicine. Patients intolerant of colchicine during the screening period are considered screen failures and not enrolled. Prophylactic administration of colchicine continues for 7 days after the last dose of study medication.

Eligible patients report to the study center early morning on Days −1, 7, 14, and 21 (or the afternoon/evening the day before). Serial blood samples are collected on Days −1, 7, 14, and 21. 24-hour urine collection is obtained on Days −1, 7, 14, and 21. Spot urine assessments also obtained on Day −1.

Study medication is administered to each patient and taken orally every morning with approximately 240 mL of water, at approximately the same time of the morning every day, and approximately 30 minutes after finishing a full breakfast (approximately 650 Kcal and 35% fat); the breakfast is standardized on Days −1 to 21.

Number of Patients

Up to 24 patients enrolled in 2 panels (up to 12 patients per panel).

Dosage and Administration

Drug 1 Capsules 100 mg and febuxostat tablets 40 mg are provided for the study. Colchicine (Colcrys®, URL Pharma) is provided by the study site.

Drug 1, febuxostat and Colchicine are taken orally.

Duration of Treatment

The duration of participation per patient is approximately 49 days, including up to a 21 day

Screening Period, 21 days of study treatment and 7 days of post-treatment colchicine prophylaxis.

Inclusion Criteria
Patient is male or post-menopausal or surgically sterile female.
Patient is 18-80 years of age, inclusive.
Patient meets one or more criteria for the diagnosis of gout as per the American Rheumatism Association (ARA) Criteria for the Classification of Acute Arthritis of Primary Gout (Appendix C).
Patient sUA ≧8 mg/dL at screening.
Patient willing and able to give informed consent and adhere to visit/protocol schedules
No clinically relevant abnormalities in blood pressure (BP), heart rate (HR), body temperature and respiratory rate as per Investigator's judgment.

Evaluation Criteria & Pharmacodynamic Evaluations

Subject urate serum levels are recorded, and any safety concerns assessed (by adverse events, clinical laboratory test results, vital signs, 12-lead ECGs, and physical examinations). The analysis of pharmacodynamic data includes all study participants receiving study drug and demonstrating evaluable data. Urate serum concentrations are evaluated for treatment- and/or time-dependent changes relative to baseline (Day −1), and may be expressed both in standard units (mg/dL) and as changes from baseline (e.g., percent change, absolute change, maximal change, and time or day of maximal change).

### Example 24

Drug 1 was tested according to the clinical trial protocol described in Example 23.

Drug 1 in combination with febuxostat was well tolerated, with no SAEs, deaths or discontinuations due to adverse events.

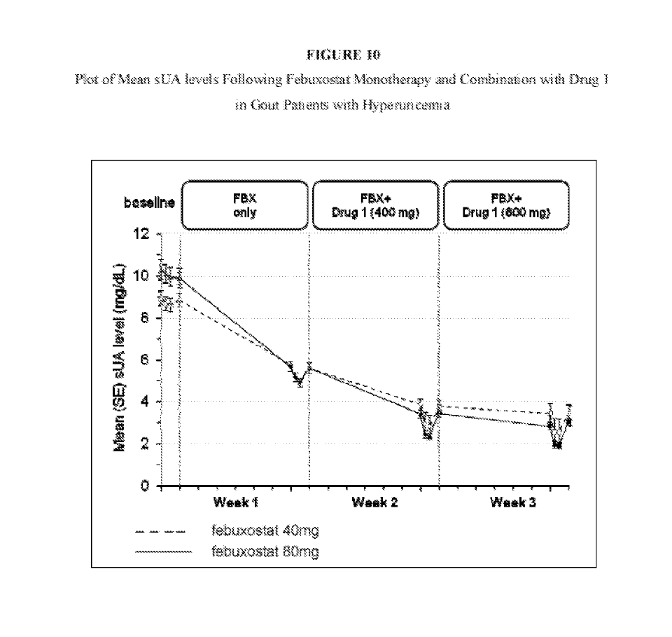

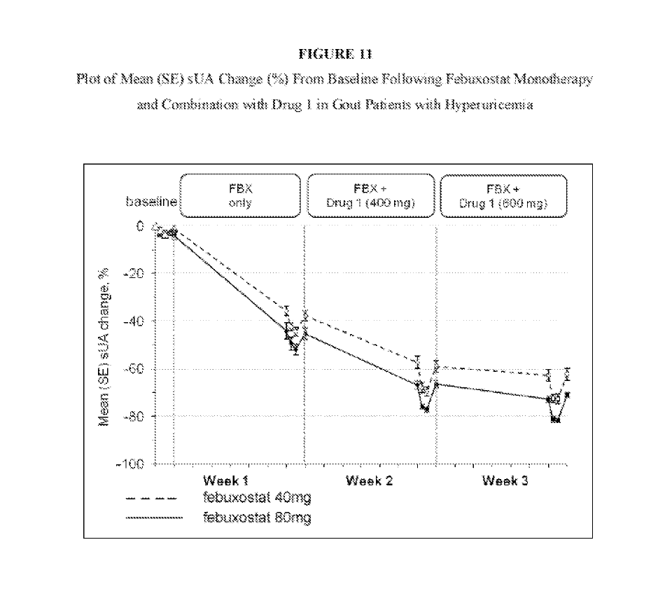

Serum urate levels were measured and absolute and % reduction levels from baseline calculated. Mean levels, mean absolute reductions (mg/dL, all data SE) and % change results are presented in the table below and in FIGS. 10 and 11.

| FBX | | | | Combination | |
|---|---|---|---|---|---|
| dose | | | | with Drug 1 | |
| (mg) | | Baseline | FBX alone | 400 mg | 600 mg |
| 40 | Mean sUA level | 8.9 ± 0.30 | 5.6 ± 0.24 | 3.8 ± 0.30 | 3.4 ± 0.31 |
| | Mean absolute | 0 | −3.2 ± 0.21 | −5.1 ± 0.26 | −5.6 ± 0.23 |
| | sUA reduction | | | | |
| | Mean sUA % | 0 | −35.7 ± 2.0 | −57.3 ± 2.6 | −62.8 ± 2.5 |
| | change | | | | |
| 80 | Mean sUA level | 10.4 ± 0.47 | 5.7 ± 0.29 | 3.4 ± 0.17 | 2.8 ± 0.17 |
| | Mean absolute | 0 | −4.6 ± 0.51 | −6.9 ± 0.44 | −7.5 ± 0.39 |
| | sUA reduction | | | | |
| | Mean sUA % | 0 | −44.2 ± 3.5 | −66.8 ± 1.7 | −72.8 ± 1.4 |
| | change | | | | |

% Response rate for sUA falling below 6, 5 or 4 mg/dL following febuxostat (FBX) monotherapy and in combination with Drug 1, are presented in the table below.

| FBX dose | | FBX | FBX + Drug 1 | |
|---|---|---|---|---|
| (mg) | Baseline | alone | 400 mg | 600 mg |
| 40 | | | | |
| (<6 mg/dL) | 0% | 67% | 100% | 100% |
| (<5 mg/dL) | 0% | 17% | 75% | 100% |
| (<4 mg/dL) | 0% | 0% | 50% | 64% |
| (<3 mg/dL) | 0% | 0% | 25% | 45% |
| 80 | | | | |
| (<6 mg/dL) | 0% | 56% | 100% | 100% |
| (<5 mg/dL) | 0% | 22% | 100% | 100% |
| (<4 mg/dL) | 0% | 0% | 89% | 100% |
| (<3 mg/dL) | 0% | 0% | 22% | 56% |

### II. Pharmaceutical Compositions Comprising a Compound of Formula (I), at Least One Pharmaceutically Acceptable Carrier and Allopurinol or Febuxostat

### Example 25

### Pharmaceutical Composition Comprising 2-(5-bromo-4-(4-cyclopropylnaphthalen-1-yl)-4H-1,2,4-triazol-3-ylthio)acetic acid and Allopurinol

### Example 25A

### Pharmaceutical Composition Comprising 2-(5-bromo-4-(4-cyclopropylnaphthalen-1-yl)-4H-1,2,4-triazol-3-ylthio)acetic acid (400 mg) and Allopurinol (300 mg)

Tablets were prepared by first granulating each drug substance separately. Water and binder solution (10% w/w Hypromellose E5) were added to 2-(5-bromo-4-(4-cyclopropylnaphthalen-1-yl)-4H-1,2,4-triazol-3-ylthio)acetic acid (403.2 mg), hypromellose (5.4 mg), microcrystalline cellulose (18.9 mg), lactose monohydrate (50.4 mg), and croscarmellose sodium (27.0 mg) while mixing in a high shear granulator (Key vertical granulator). The wet granules were dried using a fluid bed dryer and passed through a sieve.

Separately, water and binder solution (10% w/w Hypromellose E5) were added to allopurinol (300.0 mg), hypromellose (4.7 mg), microcrystalline cellulose (8.2 mg), lactose monohydrate (17.0 mg), and croscarmellose sodium (11.7 mg) while mixing in a high shear granulator (Key vertical granulator). The wet granules were dried in a vacuum oven and passed through a sieve.

The dry, sieved 2-(5-bromo-4-(4-cyclopropylnaphthalen-1-yl)-4H-1,2,4-triazol-3-ylthio)acetic acid and allopurinol-granules were combined and blended with croscarmellose sodium (46.1 mg), colloidal silicon dioxide (4.6 mg) and magnesium stearate (4.6 mg) in a tumble diffusion mixer (V-shell blender) to form a homogenous final blend which was manually compressed using a hydraulic press at a target tablet weight of 922 mg and at 2500 psi of pressure using 0.3150″×0.7087″ modified oval tooling. The resulting tablets exhibited a thickness of 6.20 mm and a crushing strength of 19 Kp. These tablets may be optionally film coated.

### Example 25B

### Pharmaceutical Composition Comprising 2-(5-bromo-4-(4-cyclopropylnaphthalen-1-yl)-4H-1,2,4-triazol-3-ylthio)acetic acid (600 mg) and Allopurinol (300 mg)

Tablets were prepared by first granulating each drug substance separately. Water and binder solution (10% w/w Hypromellose E5) were added to 2-(5-bromo-4-(4-cyclopropylnaphthalen-1-yl)-4H-1,2,4-triazol-3-ylthio)acetic acid-free acid (604.8 mg), hypromellose (8.1 mg), microcrystalline cellulose (28.4 mg), lactose monohydrate (75.6 mg), and croscarmellose sodium (40.5 mg) while mixing in a high shear granulator (Key vertical granulator). The wet granules were dried using a fluid bed dryer and passed through a sieve.

Separately, water and binder solution (10% w/w Hypromellose E5) were added to allopurinol (300.0 mg), povidone (4.7 mg), microcrystalline cellulose (8.2 mg), lactose monohydrate (17.0 mg), and croscarmellose sodium (11.7 mg) while mixing in a high shear granulator (Key vertical granulator). The wet granules were dried in a vacuum oven and passed through a sieve.

The dry, sieved 2-(5-bromo-4-(4-cyclopropylnaphthalen-1-yl)-4H-1,2,4-triazol-3-ylthio)acetic acid and allopurinol-granules were combined and blended with croscarmellose sodium (60.0 mg), colloidal silicon dioxide (6.0 mg) and magnesium stearate (6.0 mg) in a tumble diffusion mixer (V-shell blender) to form a homogenous final blend which was manually compressed using a hydraulic press at a target tablet weight of 1200 mg and at 3000 psi of pressure using 0.3937″×0.7086″ modified oval tooling. The resulting tablets exhibited a thickness of 8.12 mm and a crushing strength of 27 Kp. These tablets may be optionally film coated.

| | | mg/ | | mg/ | |
|---|---|---|---|---|---|
| Ingredient | Grade | tablet | % | tablet | % |
| INTRAGRANULAR | | | | | |
| Allopurinol | 100 | 300.0 | 32.54% | 300.0 | 25.00% |
| Povidone | K30 | 4.7 | 0.51% | 4.7 | 0.39% |
| Microcrystalline | Avicel | 8.2 | 0.89% | 8.2 | 0.68% |
| Cellulose | PH-101 | | | | |
| Lactose Monohydrate | Foremost 312 | 17.0 | 1.84% | 17.0 | 1.42% |
| Croscarmellose | AcDiSol | 11.7 | 1.27% | 11.7 | 0.98% |
| Sodium | | | | | |
| 10% PVP K30 | E5 Premium | 9.4 | 1.02% | 9.4 | 0.78% |
| | LV | | | | |
| Water | | | | | |
| Granulation Total | | 351.0 | 38.07% | 351.0 | 29.25% |
| INTRAGRANULAR | | | | | |
| 2-(5-bromo-4-(4- | 99.2* | 403.2 | 43.7% | 604.8 | 50.4% |
| cyclopropylnaphthalen- | | | | | |
| 1-yl)-4H-1,2,4-triazol- | | | | | |
| 3-ylthio)acetic acid | | | | | |
| Hypromellose | E5 Premium | 5.4 | 0.6% | 8.1 | 0.7% |
| | LV | | | | |
| Microcrystalline | Avicel | 18.9 | 2.0% | 28.4 | 2.4% |
| Cellulose | PH-101 | | | | |
| Lactose Monohydrate | Foremost 312 | 50.4 | 5.5% | 75.6 | 6.3% |
| Croscarmellose Sodium | AcDiSol | 27.0 | 2.9% | 40.5 | 3.4% |
| 10% HPMC E5 | E5 Premium | 10.8 | 1.2% | 16.2 | 1.4% |
| | LV | | | | |
| Water | | | | | |
| Granulation Total | | 515.7 | 55.94% | 773.6 | 64.47% |
| EXTRAGRANULAR | | | | | |
| Croscarmellose Sodium | AcDiSol | 46.1 | 5.0% | 60.0 | 5.0% |
| Colloidal Silicon | CabOSil | 4.6 | 0.5% | 6.0 | 0.5% |
| Dioxide | M5P | | | | |
| Magnesium Stearate | 2257, Veg | 4.6 | 0.5% | 6.0 | 0.5% |
| | source | | | | |
| Total | | 922.0 | 100.0% | 1200.0 | 100% |

*2-(5-bromo-4-(4-cyclopropylnaphthalen-1-yl)-4H-1,2,4-triazol-3-ylthio)acetic acid contained 0.64% impurities, 0.06% water, 0.15% ethyl acetate

### Example 26

### Pharmaceutical Composition Comprising 2-(5-bromo-4-(4-cyclopropylnaphthalen-1-yl)-4H-1,2,4-triazol-3-ylthio)acetic acid and Febuxostat

### Example 26A

### Pharmaceutical Composition Comprising 2-(5-bromo-4-(4-cyclopropylnaphthalen-1-yl)-4H-1,2,4-triazol-3-ylthio)acetic acid (400 mg) and Febuxostat (80 mg)

Water and binder solution (10% Hypromellose E5) were added to a mixture of 2-(5-bromo-4-(4-cyclopropylnaphthalen-1-yl)-4H-1,2,4-triazol-3-ylthio)acetic acid-free acid (403.2 mg), hypromellose (5.4 mg), microcrystalline cellulose (18.9 mg), lactose monohydrate (50.4 mg), and croscarmellose sodium (27.0 mg) while mixing in a high shear granulator (i.e. Key vertical granulator). The resulting wet granules were dried using a fluid bed dryer and passed through a sieve. Febuxostat (80.0 mg) was passed through a 40-mesh screen and combined with croscarmellose sodium (31.7 mg), colloidal silicon dioxide (3.2 mg), and magnesium stearate (3.2 mg). The 2-(5-bromo-4-(4-cyclopropylnaphthalen-1-yl)-4H-1,2,4-triazol-3-ylthio)acetic acid granulation and the febuxostat mixture were blended in a tumble diffusion mixer (V-shell blender) to form a homogenous final blend suitable for compression into tablets.

Tablets were manually compressed using a hydraulic press at a target tablet weight of 634 mg and at 2000 psi of pressure using 0.2930″×0.6630″ modified oval tooling. The resulting tablets exhibited a thickness of 5.76 mm and a crushing strength of approximately 12 Kp. These tablets may be optionally film coated.

### Example 26B

### Pharmaceutical Composition Comprising 2-(5-bromo-4-(4-cyclopropylnaphthalen-1-yl)-4H-1,2,4-triazol-3-ylthio)acetic acid (600 mg) and Febuxostat (80 mg)

Water and binder solution (10% Hypromellose E5) were added to a mixture of 2-(5-bromo-4-(4-cyclopropylnaphthalen-1-yl)-4H-1,2,4-triazol-3-ylthio)acetic acid (604.8 mg), hypromellose (8.1 mg), microcrystalline cellulose (28.4 mg), lactose monohydrate (75.6 mg), and croscarmellose sodium (40.5 mg) while mixing in a high shear granulator (i.e. Key vertical granulator). The resulting wet granules were dried using a fluid bed dryer and passed through a sieve. Febuxostat (80.0 mg) was passed through a 40-mesh screen and combined with croscarmellose sodium (45.3 mg), colloidal silicon dioxide (4.5 mg), and magnesium stearate (4.5 mg). The 2-(5-bromo-4-(4-cyclopropylnaphthalen-1-yl)-4H-1,2,4-triazol-3-ylthio)acetic acid granulation and the febuxostat mixture were blended in a tumble diffusion mixer (V-shell blender) to form a homogenous final blend suitable for compression into tablets.

Tablets were manually compressed using a hydraulic press at a target tablet weight of 905 mg and at 2500 psi of pressure using 0.3070″×0.6940″ modified oval tooling. The resulting tablets exhibited a thickness of 6.91 mm and a crushing strength of approximately 23 Kp. These tablets may be optionally film coated.

| Ingredient | Grade | mg/tablet | % | mg/tablet | % |
|---|---|---|---|---|---|
| INTRAGRANULAR | | | | | |
| 2-(5-bromo-4-(4- | 99.2* | 403.2 | 63.6% | 604.8 | 66.8% |
| cyclopropylnaphthalen-1- | | | | | |
| yl)-4H-1,2,4-triazol-3- | | | | | |
| ylthio)acetic acid | | | | | |
| Hypromellose | E5 Premium LV | 5.4 | 0.9% | 8.1 | 0.9% |
| Avicel | PH-101 | 18.9 | 3.0% | 28.4 | 3.1% |
| Lactose Monohydrate | Foremost 312 | 50.4 | 7.9% | 75.6 | 8.4% |
| Croscarmellose Na | AcDiSol | 27.0 | 4.3% | 40.5 | 4.5% |
| 10% HPMC E5 | E5 Premium LV | 10.8 | 1.7% | 16.2 | 1.8% |
| Water | | | | | |
| Granulation Total | | 515.7 | 81.34% | 773.6 | 85.48% |
| EXTRAGRANULAR | | | | | |
| Febuxostat | | 80.0 | 12.62% | 80.0 | 8.84% |
| Croscarmellose Na | AcDiSol | 31.7 | 5.0% | 45.3 | 5.0% |
| Colloidal Silicon Dioxide | CabOSil M5P | 3.2 | 0.5% | 4.5 | 0.5% |
| Magnesium Stearate | 2257, Veg source | 3.2 | 0.5% | 4.5 | 0.5% |
| Total | | 634.0 | 100.0% | 905.0 | 100% |

*2-(5-bromo-4-(4-cyclopropylnaphthalen-1-yl)-4H-1,2,4-triazol-3-ylthio)acetic acid contained 0.64% impurities, 0.06% water, 0.15% ethyl acetate

1-20. (canceled)

21. A pharmaceutical composition, comprising
i) about 100 mg to about 1000 mg of allopurinol;
ii) about 100 mg to about 400 mg of a compound of formula: 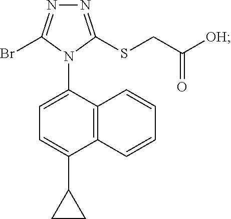 [SMILES: O=C(O)CSc1nnc(Br)n1-c1ccc(C2CC2)c2ccccc12]
and
iii) at least one pharmaceutically acceptable carrier.

22. The pharmaceutical composition of claim 21, comprising
i) about 100 mg, about 150 mg, about 200 mg, about 300 mg, about 400 mg, about 600 mg, or about 800 mg of allopurinol;
ii) about 100 mg, about 200 mg or about 400 mg of a compound of formula: 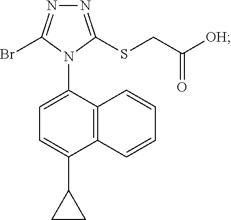 [SMILES: O=C(O)CSc1nnc(Br)n1-c1ccc(C2CC2)c2ccccc12]
and
iii) at least one pharmaceutically acceptable carrier.

23. The pharmaceutical composition of claim 21, comprising
i) about 300 mg of allopurinol;
ii) about 200 mg or about 400 mg of a compound of formula: 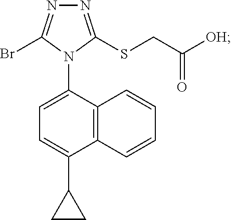 [SMILES: O=C(O)CSc1nnc(Br)n1-c1ccc(C2CC2)c2ccccc12]
and
iii) at least one pharmaceutically acceptable carrier.

24. The pharmaceutical composition of claim 21, comprising
i) about 200 mg of allopurinol;
ii) about 200 mg or about 400 mg of a compound of formula: 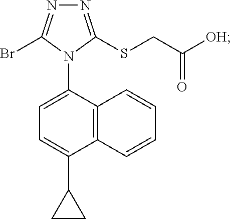 [SMILES: O=C(O)CSc1nnc(Br)n1-c1ccc(C2CC2)c2ccccc12]
and
iii) at least one pharmaceutically acceptable carrier.

25. A pharmaceutical composition, comprising
i) about 20 mg to about 200 mg of febuxostat;
ii) about 100 mg to about 400 mg of a compound of formula: 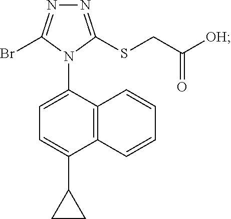 [SMILES: O=C(O)CSc1nnc(Br)n1-c1ccc(C2CC2)c2ccccc12]
and
iii) at least one pharmaceutically acceptable carrier.

26. The pharmaceutical composition of claim 25, comprising
i) about 20 mg, about 40 mg, about 60 mg, about 80 mg, or about 120 mg of febuxostat;
ii) about 100 mg, about 200 mg, or about 400 mg of a compound of formula: 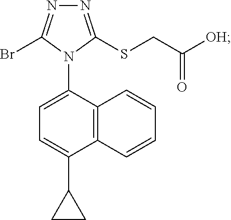 [SMILES: O=C(O)CSc1nnc(Br)n1-c1ccc(C2CC2)c2ccccc12]
and
iii) at least one pharmaceutically acceptable carrier.

27. A method of treating or preventing hyperuricemia or gout in a subject, comprising administering to the subject:
i) allopurinol, febuxostat, or combinations thereof; and
ii) a compound of formula (I): 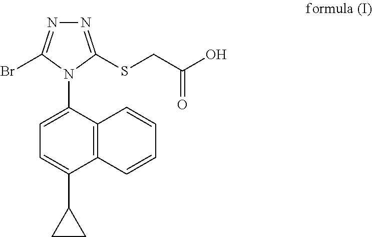 [SMILES: O=C(O)CSc1nnc(Br)n1-c1ccc(C2CC2)c2ccccc12]
wherein the gout or hyperuricemia is refractory, non-responsive, or resistant to allopurinol monotherapy, febuxostat monotherapy, PNP-inhibitor monotherapy, probenecid monotherapy, tranilast monotherapy, sulfinpyrazone monotherapy, losartan monotherapy, fenofibrate monotherapy, and/or benzbromarone monotherapy.

28. The method of claim 27, comprising administering from about 100 mg to about 1000 mg of the compound of formula (I).

29. The method of claim 27, comprising administering from about 100 mg to about 1000 mg of allopurinol.

30. The method of claim 27, wherein
prior to administration of allopurinol and the compound of formula (I), the subject has received treatment with allopurinol; and
wherein the allopurinol treatment does not decrease serum uric acid levels of the subject below about 6 mg/dL; and
wherein after administration of allopurinol and the compound of formula (I), serum uric acid levels of the subject decrease below about 6 mg/dL.

31. The method of claim 27, wherein
prior to administration febuxostat and the compound of formula (I), the subject has received treatment with febuxostat; and
wherein the febuxostat treatment does not decrease serum uric acid levels of the subject below about 6 mg/dL; and
wherein after administration of febuxostat and the compound of formula (I), serum uric acid levels of the subject decrease below about 6 mg/dL.

32. A method of reducing serum uric acid levels in a subject suffering from hyperuricemia, comprising administering to the subject a compound of formula: 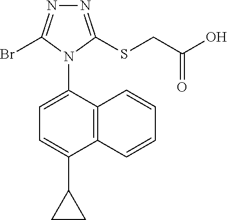 [SMILES: O=C(O)CSc1nnc(Br)n1-c1ccc(C2CC2)c2ccccc12]
wherein after administration the subject has:
a serum uric acid level less than about 6.0 mg/dL; and
a creatinine clearance rate below about 60 mL/minute.

33. The method of claim 32, wherein prior to administration the subject has a serum uric acid level greater than about 6.0 mg/dL.

34. The method of claim 32, wherein after administration the subject has a creatinine clearance rate of from about 30 mL/minute to about 60 mL/minute.

35. The method of claim 32, further comprising administering allopurinol.
